(19) United States
(12) Patent Application Publication
(10) Pub. No.: US 2025/0346933 A1
(43) Pub. Date: Nov. 13, 2025

(54) FUNGAL CELLS FOR PRODUCTION OF FATTY ACIDS AND FATTY ACID-DERIVED PRODUCTS

(71) Applicant: Melt&Marble AB, Göteborg (SE)

(72) Inventors: Anastasia KRIVORUCHKO, Göteborg (SE); Jens NIELSEN, Hellerup (DK); Florian DAVID, Göteborg (SE); Tao YU, Shenzhen (CN)

(21) Appl. No.: 19/276,384

(22) Filed: Jul. 22, 2025

Related U.S. Application Data
(63) Continuation of application No. 18/330,457, filed on Jun. 7, 2023, now Pat. No. 12,398,409.
Application No. 18/330,457 is a continuation of application No. 16/830,854, filed on Mar. 26, 2020, now Pat. No. 11,692,209.
(60) Provisional application No. 62/824,398, filed on Mar. 27, 2019.

(51) Int. Cl. C12P 7/6409; C12N 1/16; C12N 9/00

(52) U.S. Cl. CPC C12P 7/6409; C12N 1/16; C12N 9/93; C12Y 604/01001; C12Y 604/01002

(57) ABSTRACT

A yeast cell for the production of fatty acids and/or fatty acid-derived products is genetically modified for overexpression of one or more endogenous yeast genes selected from the group consisting of ADP-ribosylation factor-binding protein, phosphatidylinositol 4,5-bisphosphate 5-phosphatase INP54, NADP-dependent isocitrate dehydrogenase IDP3, mitochondrial phosphatidylglycerophosphatase GEP4, triglyceride lipase TGL1, long chain fatty acyl-CoA synthetase FAA3, lipid phosphate phosphatase LPP1, and variants of such endogenous yeast genes. The yeast cell produces higher amount of fatty acids and/or fatty acid-derived products compared to a yeast cell not overexpressing the one or more endogenous yeast genes.

The Sequence Listing submitted herewith entitled Jun.-7-2023-Sequence-Listing.XML, created Jul. 21, 2025 and having a size of 63,499 bytes, is incorporated herein by reference.

### CROSS-REFERENCE TO RELATED APPLICATION

This application is based on and claims priority under 35 USC 119 to U.S. Provisional application No. 62/824,398, filed on Mar. 27, 2019, in the U.S. Patent and Trademark Office, and the entire contents thereof are incorporated herein by reference. This application also claims priority under 35 USC 120 to U.S. application Ser. No. 16/830,854 filed Mar. 26, 2020, and the entire contents thereof are incorporated herein by reference, and to U.S. application Ser. No. 18/330,457 filed Jun. 7, 2023, and the entire contents thereof are incorporated herein by reference.

### TECHNICAL FIELD

The present invention relates generally to the development of genetically engineered microorganisms. More specifically, the invention relates to fungal cells able to produce fatty acids and/or fatty acid-derived products in an economic fashion.

### BACKGROUND

Fatty acids are carboxylic acids with a long aliphatic chain that is either saturated or unsaturated. Fatty acids and their derived products, e.g., fatty alcohols, fatty acid esters, etc., have numerous commercial applications including as surfactants, lubricants, plasticizers, solvents, emulsifiers, emollients, thickeners, flavors, pesticides, cosmetics, nutraceuticals and fuels. Current technologies for producing fatty acids and fatty acid-derived products are typically via extraction from plant or animal sources, such as coconut, palm, palm kernel, tallow and lard. However, due to concerns regarding the sustainability of these sources, as well as increasing demands for specialty fatty acids that cannot be easily derived from natural sources, alternative production methods are needed. For example, research efforts have focused on production of fatty acids via microbial fermentation (Pfleger et al., 2015). In addition, recent advances in genetic and metabolic engineering have allowed for precise manipulation of the microbial metabolism to produce tailor-made products. Other advantages of these production platforms include environmental friendliness, scalability, geographical independence, and cost effectiveness. Microbial fatty acid biosynthesis has attracted much attention for production of oleochemicals and biofuels. Engineering of central metabolism and fatty acid biosynthesis enabled fatty acid overproduction in Escherichia coli, Saccharomyces cerevisiae, and Yarrowia lipolytica. However, the production titer and yield need to be further enhanced to enable industrial production using new strategies.

There is therefore still a need for techniques for the production of fatty acids and/or fatty acid-derived products in yeast cells in an efficient way.

### SUMMARY

It is a general objective to provide improved production of fatty acids and/or fatty acid-derived products in fungal cells.

The present invention provides a genetically engineered fungal cell, preferably a yeast cell, which comprises genetic modifications that allow increased production of fatty acids and/or fatty acid-derived products. The fungal cell is genetically modified for overexpression of an acetyl-CoA carboxylase and a pyruvate carboxylase.

The yeast Saccharomyces cerevisiae is a very important cell factory as it is already widely used for production of biofuels, chemicals and pharmaceuticals, and there is therefore much interest in developing platform strains of this yeast that can be used for production of a whole range of different products. It is, however, a problem that such a platform cell factory for efficient production of fatty acids and fatty acid-derived products is not as efficient as needed for good industrial application. This invention involves a multiple gene modification approach of the yeast to generate a stable and scalable platform for production fatty acids and fatty acid-derived products.

The present invention relates to a fungal cell and methods as defined in the independent claims. Further embodiments of the invention are defined in the dependent claims.

### BRIEF DESCRIPTION OF THE DRAWINGS

The embodiments, together with further objects and advantages thereof, may best be understood by making reference to the following description taken together with the accompanying drawings, in which:

FIG. 1: Schematic illustration of various modifications for increased fatty acid production. Engineering targets include tesA, the truncated E. coli thioesterase; MmACL, ATP:citrate lyase from Mus musculus; RtME, cytosolic NADP+-dependent malic enzyme from Rhodosporidium toruloides; MDH3, endogenous malate dehydrogenase with removed peroxisomal signal; mPYC1, mitochondria-targeted pyruvate carboxylase; CTP1, citrate transporter and RtFAS, fatty acid synthase from Rhodosporidium toruloides. For free fatty acid (FFA) production, fatty acyl-CoA synthetase encoding genes FAA1 and FAA4, and fatty acyl-CoA oxidase encoding gene POX1 were disrupted. Additional engineering targets include MPCox, overexpressed endogenous mitochondrial pyruvate carrier (MPC1 and MPC3); RtCIT1, citrate synthase from Rhodosporidium toruloides; ScCIT1, citrate synthase from Saccharomyces cerevisiae; AnACL, ATP:citrate lyase from Aspergillus nidulans; PDA1, pyruvate dehydrogenase alpha; E3 (LPD1), dihydrolipoamide dehydrogenase; PGI1, phosphoglucose isomerase; ZWF1, cytoplasmic glucose-6-phosphate dehydrogenase; GND1, the isoform 1 of phosphogluconate dehydrogenase; TKL1, transketolase 1; TAL1, transaldolase 1 and IDH2, subunit 2 of mitochondrial NAD(+)-dependent isocitrate dehydrogenase. Native PYC1 (pyruvate carboxylase 1), YHM2 (citrate and oxoglutarate carrier protein 2), IDP2 (cytosolic NADP-specific isocitrate dehydrogenase 2) and ACC1 (acetyl-CoA carboxylase 1) were overexpressed. For the abolishment of ethanol production, pyruvate carboxylase encoding genes PDC1, PDC5 and PDC6 were disrupted. To fine tune gene expression, the promoter of PGI1 was replaced by the promoters of ISU1 (IScU homolog 1), ATP14 (ATP synthase 14), QCR10 (ubiQuinol-cytochrome C oxidoReductase 10), COX9 (Cytochrome c Oxidase 9), NAT1 (N-terminal AcetylTransferase 1), and HXT1 (Low-affinity glucose transporter 1), and the promoter of IDH2 was replaced by the promoters of INH1 (Protein that inhibits ATP hydrolysis by the F1F0-ATP synthase 1), SDH4 (Membrane anchor subunit of succinate dehydrogenase 4), ATP5 (ATP synthase 5), GSY2 (Glycogen synthase 2), GSP2 (GTP binding protein 2), RBK1 (RiBoKinase 1), and HXT1.

FIG. 2: Metabolic engineering for enhancing the supply of the cytosolic acetyl-CoA. (A) Schematic illustration of the subcellular flux trafficking and engineering targets. (B) FFA production obtained with engineered strains in shake flasks after 72 h cultivation at 200 rpm, 30° C. on 30 g/L glucose. All data is presented as mean±SD of biological triplicates.

FIGS. 3A-3C: Fine-tuning the pentose phosphate pathway (PPP), tricarboxylic acid cycle (TCA) cycle and glycolysis for FFA production. (FIG. 3A) Schematic illustration of metabolic connections between glycolysis, TCA cycle and PPP. Pushing carbon flux into PPP for improving FFA production by tuning PGI1 and IDH2 expression. Fine tuning of PGI1 (FIG. 3B) and IDH2 (FIG. 3C) improved FFA production up to 60%. The strains were cultivated in shake flasks for 80 h at 200 rpm, 30° C. with glucose feed beads corresponding to 30 g/L glucose. All data represent the mean±s.d. of biological triplicates.

FIG. 4: FFA production was further improved by growth decoupling. Limited cell growth by downregulation of essential genes and nitrogen restriction improved FFA production. The strains were cultivated in shake flasks for 80 h at 200 rpm, 30° C. with glucose feed beads (corresponding to 30 g/L glucose). All data represent the mean±SD of biological triplicates.

FIG. 5: FFA production was further improved by growth decoupling. (A) Fed-batch fermentation of strain TY36 with glucose limited and nitrogen restriction. Time courses of FFA titers and end point are shown. Overall FFA production at the end of fermentation is 35 g/L. (B) Time courses of DCW (upper curve) and consumed glucose (lower curve) during the fermentation are shown.

FIGS. 6A-6C: Rewiring yeast from alcoholic fermentation to fatty acid production. (FIG. 6A) The production profile of wild-type S. cerevisiae (CEN.PK113-5D), an evolved wild-type pyruvate dexarboxylase (PDC)-negative strain and the evolved TY53 strain. Strains were cultured in shake flasks at 200 rpm, 30° C. on 30 g/L glucose. All data represent the mean±SD of biological triplicates. (FIG. 6B) Free fatty acid production in fed-batch cultures of the evolved PDC-negative yeast with glucose limitation and nitrogen restriction. Circle indicates overall free fatty acid production at the end of fermentation. (FIG. 6C) Time-course for glucose consumption and dry cell weight accumulation during fed-batch fermentation of evolved pyruvate decarboxylase-negative yeast.

FIG. 7: Venn diagram summarizing the intersection among mutations accumulated in the evolved strains TY53 isolated from three distinct evolution experiments.

FIG. 8: Growth curves for the three evolved TY53 populations. The strains were cultured in shake flasks at 200 rpm, 30° C. with 20 g/L glucose. All data represent the mean±SD of biological triplicates.

FIG. 9: Activity of pyruvate kinase (PK). (A) The overall activity of PK in the evolved strains was downregulated. Fructose-1,6-bisphosphate (FBP) was added as an activator of PYK1. (B) The activity of PYK2 in the evolved strains was increased. Here, no FBP was added as an activator.

FIG. 10: The evolved phenotype was abolished by expression of PYK1. The strains were precultured in shake flasks at 200 rpm, 30° C. with 20 g/L glucose for 3 days to remove intracellular stores of C2 metabolites, then subcultured in shake flasks at 200 rpm, 30° C. with 20 g/L glucose for measurement of optical density at 600 nm (OD₆₀₀). All data represent the mean±SD of biological triplicates.

FIG. 11: Downregulation of FBP-sensitive and upregulation of FBP-insensitive pyruvate kinase enabled the growth of PDC-negative strain in high concentration of glucose. The strains were cultured in shake flasks at 200 rpm, 30° C. with 20 g/L glucose and 0.5% (v/v) ethanol. All data represent the mean±SD of biological triplicates. The activity of GSP1 promoter is 6-fold of native PYK2 promoter. The activity of MCM1 promoter is 3-fold of native PYK2 promoter. The PYK1 gene was deleted in TY53, then a PYK2 gene under the promoter of GSP1 or MCM1 in a low-copy plasmid was introduced into the cell to get the strain TY53 pyk1ΔGSP1p-PYK2 or TY53pyk1ΔMCM1p-PYK2.

FIG. 12: Effect of individual overexpression of GGA2, INP54, EPT1, FAA1, IDP3, MPP6, GEP4, ACP1, ORM1, MCR1, TGL1, YFT2, SPO7, FAA3, LPP1 and RTC3 on production of fatty acids. IMX581 is the control strain (not overexpressing any of the aforementioned genes). Strains were cultivated at 30° C. and 200 rpm and samples were taken after 48 hours. All numbers are an average of three replicates. Dashed horizontal line indicates production of control strain for easier comparison.

FIG. 13: Effect of individual overexpression of GGA2, INP54, EPT1, FAA1, IDP3, MPP6, GEP4, ACP1, ORM1, MCR1, TGL1, YFT2, SPO7, FAA3, LPP1 and RTC3 on fatty acid composition. IMX581 is the control strain (not overexpressing any of the aforementioned genes). Strains were cultivated at 30° C. and 200 rpm. Samples were taken after 48 hours of cultivation. All numbers are an average of three replicates. C16:0 is palmitic acid, C16:1 is palmitoleic acid, C18:0 is stearic acid, C18:1 is oleic acid. Dashed horizontal lines indicate production of the respective fatty acid by the control strain for easier comparison.

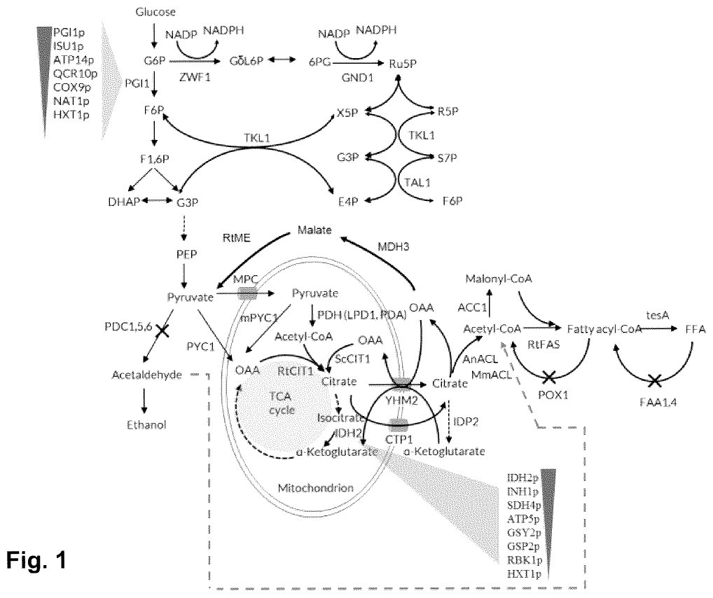

### DETAILED DESCRIPTION

The present invention now will be described hereinafter with reference to the accompanying drawings and examples, in which embodiments of the invention are shown. This description is not intended to be a detailed catalogue of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations and variations thereof.

Unless otherwise defined herein, scientific and technical terms used herein will have the meanings that are commonly understood by those of ordinary skill in the art.

Generally, nomenclatures used in connection with techniques of biochemistry, enzymology, molecular and cellular biology, microbiology, genetics and protein and nucleic acid chemistry and hybridization, described herein, are those well-known and commonly used in the art.

Conventional methods and techniques mentioned herein are explained in more detail, for example, in Molecular Cloning, a laboratory manual [second edition] Sambrook et al. Cold Spring Harbor Laboratory, 1989, for example in Sections 1.21 “Extraction And Purification Of Plasmid DNA”, 1.53 “Strategies For Cloning In Plasmid Vectors”, 1.85 “Identification Of Bacterial Colonies That Contain Recombinant Plasmids”, 6 “Gel Electrophoresis Of DNA”, 14 “In vitro Amplification Of DNA By The Polymerase Chain Reaction”, and 17 “Expression Of Cloned Genes In Escherichia coli” thereof.

Enzyme Commission (EC) numbers (also called “classes” herein), referred to throughout this specification, are according to the Nomenclature Committee of the International Union of Biochemistry and Molecular Biology (NC-IUBMB) in its resource “Enzyme Nomenclature” (1992, including Supplements 6-17) available, for example, as “Enzyme nomenclature 1992: recommendations of the Nomenclature Committee of the International Union of Biochemistry and Molecular Biology on the nomenclature and classification of enzymes”, Webb, E. C. (1992), San Diego: Published for the International Union of Biochemistry and Molecular Biology by Academic Press (ISBN 0-12-227164-5). This is a numerical classification scheme based on the chemical reactions catalyzed by each enzyme class.

The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

Throughout the description and claims of this specification, the words “comprise” and “contain” and variations of the words, for example “comprising” and “comprises”, mean “including but not limited to” and do not exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

As used herein, the transitional phrase “consisting” essentially of means that the scope of a claim is to be interpreted to encompass the specified materials or steps recited in the claim and those that do not materially affect the basic and novel characteristic(s) of the claimed invention. Thus, the term “consisting essentially of when used in a claim of this invention is not intended to be interpreted to be equivalent to “comprising.”

To facilitate understanding of the invention, a number of terms are defined below.

As used herein, the term “fatty acid” refers to a carboxylic acid with a long aliphatic chain, composed of 4 to 40 carbons, which is either saturated or unsaturated. An unsaturated fatty acid contains at least one double or triple bond within its aliphatic chain, which can occur at any position. To define the position of the double bond, the delta-x (delta(x) or A-x) nomenclature is used herein. In this nomenclature, each double bond is indicated by “delta(x)”, where the double bond is located on the x^{th} carbon-carbon bond, counting from the carboxylic acid end. A fatty acid can be either straight-chained or have branches, i.e., with one or more alkyl groups, such as methyl groups, on the carbon chain. Furthermore, a fatty acid can have additional modifications, such as hydroxylation, i.e., a hydroxy fatty acid, epoxidation, i.e., an epoxy fatty acid and/or comprise multiple, i.e., at least two, carboxylic groups, such as a dicarboxylic fatty acid. Within the cell, fatty acids can occur as free fatty acids (FFAs), fatty acyl-CoAs, fatty acyl-ACPs, fatty acids within triacylglycerols (TAGs), fatty acids within steryl esters, or fatty acids within phospholipids.

As used herein, the term “fatty acid-derived product” refers to any molecule that is created by further modification of a fatty acid in the fungal cell. Examples of fatty acid derived products include, but are not limited to fatty alcohols, fatty aldehydes, fatty acid esters, hydrocarbons, triacylglycerides, lactones and phospholipids.

The term “fatty acyl-CoA” refers to a fatty acid that is bound to a coenzyme A (CoA).

The term “fatty acyl-ACP” refers to a fatty acid that is found to an acyl carrier protein (ACP).

Also, as used herein, the terms “nucleotide sequence” “nucleic acid,” “nucleic acid molecule,” “oligonucleotide” and “polynucleotide” refer to RNA or DNA, including cDNA, a DNA fragment or portion, genomic DNA, synthetic DNA, plasmid DNA, mRNA, and anti-sense RNA, any of which can be single stranded or double stranded, linear or branched, or a hybrid thereof. Nucleic acid molecules and/or nucleotide sequences provided herein are presented herein in the 5′ to 3′ direction, from left to right and are represented using the standard code for representing the nucleotide characters as set forth in the U.S. sequence rules, 37 CFR §§ 1.821-1.825 and the World Intellectual Property Organization (WIPO) Standard ST.25.

As used herein the term “recombinant” when used means that a particular nucleic acid (DNA or RNA) is the product of various combinations of cloning, restriction, and/or ligation steps resulting in a construct having a structural coding or non-coding sequence distinguishable from endogenous nucleic acids found in natural systems.

As used herein, the term “gene” refers to a nucleic acid molecule capable of being used to produce mRNA, antisense RNA, miRNA, anti-microRNA antisense oligodeoxyribonucleotide (AMO) and the like. Genes may or may not be capable of being used to produce a functional protein or gene product. Genes can include both coding and non-coding regions, e.g., introns, regulatory elements, promoters, enhancers, termination sequences and/or 5′ and 3′ untranslated regions. A gene may be “isolated” by which is meant a nucleic acid that is substantially or essentially free from components normally found in association with the nucleic acid in its natural state. Such components include other cellular material, culture medium from recombinant production, and/or various chemicals used in chemically synthesizing the nucleic acid.

A “disrupted gene” as defined herein involves any mutation or modification to a gene resulting in a partial or fully non-functional gene and gene product. Such a mutation or modification includes, but is not limited to, a missense mutation, a nonsense mutation, a deletion, a substitution, an insertion, addition of a targeting sequence and the like. Furthermore, a disruption of a gene can be achieved also, or alternatively, by mutation or modification of control elements controlling the transcription of the gene, such as mutation or modification in a promoter, terminator and/or enhancement elements. In such a case, such a mutation or modification results in partially or fully loss of transcription of the gene, i.e., a lower or reduced transcription as compared to native and non-modified control elements. As a result a reduced, if any, amount of the gene product will be available following transcription and translation. Furthermore, disruption of a gene could also entail adding or removing a localization signal from the gene, resulting in decreased presence of the gene product in its native subcellular compartment.

The objective of gene disruption is to reduce the available amount of the gene product, including fully preventing any production of the gene product, or to express a gene product that lacks or having lower enzymatic activity as compared to the native or wild type gene product.

As used herein the term “deletion” or “knock-out” refers to a gene that is inoperative or knocked out.

The term “lowered activity” or “attenuated activity” when related to an enzyme refers to a decrease in the activity of the enzyme in its native compartment compared to a control or wild-type state. Manipulations that result in attenuated activity of an enzyme include, but are not limited to, a missense mutation, a nonsense mutation, a deletion, a substitution, an insertion, addition of a targeting sequence, removal of a targeting sequence, or the like. Furthermore, attenuation of enzyme activity can be achieved also, or alternatively, by mutation or modification of control elements controlling the transcription of the gene encoding the enzyme, such as mutation or modification in a promoter, terminator and/or enhancement elements. A cell that contains modifications that result in attenuated enzyme activity will have a lower activity of the enzyme compared to a cell that does not contain such modifications. Attenuated activity of an enzyme may be achieved by encoding a nonfunctional gene product, e.g., a polypeptide having essentially no activity, e.g., less than about 10% or even 5% as compared to the activity of the wild type polypeptide.

A “codon optimized” version of a gene refers to an exogenous gene introduced into a cell and where the codons of the gene have been optimized with regard to the particular cell. Generally, not all tRNAs are expressed equally or at the same level across species. Codon optimization of a gene sequence thereby involves changing codons to match the most prevalent tRNAs, i.e., to change a codon recognized by a low prevalent tRNA with a synonymous codon recognized by a tRNA that is comparatively more prevalent in the given cell. This way the mRNA from the codon optimized gene will be more efficiently translated. The codon and the synonymous codon preferably encode the same amino acid.

As used herein, the term “allele” refers to a variant form of a given gene. This can include a mutated form of a gene where one or more of the amino acids encoded by the gene have been removed or substituted by a different amino acid.

As used herein, the terms “peptide”, “polypeptide”, and “protein” are used interchangeably to indicate to a polymer of amino acid residues. The terms “peptide”, “polypeptide” and “protein” also includes modifications including, but not limited to, lipid attachment, glycosylation, glycosylation, sulfation, hydroxylation, γ-carboxylation of L-glutamic acid residues and ADP-ribosylation.

As used herein, the term “enzyme” is defined as a protein which catalyzes a chemical or a biochemical reaction in a cell. Usually, according to the present invention, the nucleotide sequence encoding an enzyme is operably linked to a nucleotide sequence (promoter) that causes sufficient expression of the corresponding gene in the cell to confer to the cell the ability to produce fatty acids.

As used herein, the term “open reading frame (ORF)” refers to a region of RNA or DNA encoding polypeptide, a peptide, or protein.

As used herein, the term “genome” encompasses both the plasmids and chromosomes in a host cell. For instance, encoding nucleic acids of the present disclosure which are introduced into host cells can be portion of the genome whether they are chromosomally integrated or plasmids-localized.

As used herein, the term “promoter” refers to a nucleic acid sequence which has functions to control the transcription of one or more genes, which is located upstream with respect to the direction of transcription of the transcription initiation site of the gene. Suitable promoters in this context include both constitutive and inducible natural promoters as well as engineered promoters, which are well known to the person skilled in the art. In this application, promoters are designed with a “p” in front of the gene name (e.g., “pTEF1” is the promoter of the gene TEF1).

Suitable promoters for use in eukaryotic host cells, such as yeast cells, may be the promoters of PDC, GPD1, TEF1, PGK1 and TDH. Other suitable promoters include the promoters of GAL1, GAL2, GAL10, GAL7, CUP1, HIS3, CYC1, ADH1, PGL, GAPDH, ADC1, URA3, TRP1, LEU2, TPI, AOX1 and ENO1.

As used herein, the term “promoter activity” refers to the ability of a promoter to facilitate expression of the gene lying immediately downstream of said promoter. Typical indicators of a promoter's activity include the timing of expression and level of expression of its downstream gene relative to other genes. A promoter with high or strong activity will lead to high levels of transcription of the gene lying immediately downstream of said promoter, subsequently resulting in high mRNA (and subsequently protein) levels of said gene. A promoter with weak or low activity will lead to low levels of transcription of the gene lying immediately downstream of said promoter, subsequently resulting in low mRNA levels of said gene. Promoter activity can usually be assessed by measuring the mRNA expression of its downstream gene, or by placing a reporter gene immediately downstream of a promoter and observing e.g., fluorescence or colour formation upon respective protein formation. Factors influencing the strength and activity of a promoter can include transcription factor binding (dependent on binding sites in the promoter), efficiency of recruiting RNA polymerases, environmental conditions, etc.

As used herein, the term “terminator” refers to a “transcription termination signal” if not otherwise noted. Terminators are sequences that hinder or stop transcription of a polymerase.

As used herein, “recombinant eukaryotic cells” according to the present disclose is defined as cells which contain additional copies or copy of an endogenous nucleic acid sequence or are transformed or genetically modified with polypeptide or a nucleotide sequence that does not naturally occur in the eukaryotic cells. The wildtype eukaryotic cells are defined as the parental cells of the recombinant eukaryotic cells, as used herein.

As used herein, “recombinant prokaryotic cells” according to the present disclose is defined as cells which contain additional copies or copy of an endogenous nucleic acid sequence or are transformed or genetically modified with polypeptide or a nucleotide sequence that does not naturally occur in the prokaryotic cells. The wildtype prokaryotic cells are defined as the parental cells of the recombinant prokaryotic cells, as used herein.

As used herein, the terms “increase,” “increases,” “increased,” “increasing,” “enhance,” “enhanced,” “enhancing,” and “enhancement” (and grammatical variations thereof) indicate an elevation of at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500% or more, or any range therein, as compared to a control.

As used herein, the terms “reduce,” “reduces,” “reduced,” “reduction,” “diminish,” “suppress,” and “decrease” and similar terms mean a decrease of at least about, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500% or more, or any range therein, as compared to a control.

A reduced expression of a gene as used herein involves a genetic modification that reduces the transcription of the gene, reduces the translation of the mRNA transcribed from the gene and/or reduces post-translational processing of the protein translated from the mRNA. Such genetic modification includes insertion(s), deletion(s), replacement s) or mutation(s) applied to the control sequence, such as a promoter and enhancer, of the gene. For instance, the promoter of the gene could be replaced by a less active or inducible promoter to thereby result in a reduced transcription of the gene. Also a knock-out of the promoter would result in reduced, typically zero, expression of the gene.

As used herein, the term “portion” or “fragment” of a nucleotide sequence of the invention will be understood to mean a nucleotide sequence of reduced length relative to a reference nucleic acid or nucleotide sequence and comprising, consisting essentially of and/or consisting of a nucleotide sequence of contiguous nucleotides identical or almost identical, e.g., 80%, 81%, 82%, 83%, 84%, 85%, 86%, 87%, 88%, 89%, 90%, 91%, 92%, 93%, 94%, 95%, 96%. 98%, 99% identical, to the reference nucleic acid or nucleotide sequence. Such a nucleic acid fragment or portion according to the invention may be, where appropriate, included in a larger polynucleotide of which it is a constituent.

Different nucleic acids or proteins having homology are referred to herein as “homologues.” The term homologue includes homologous sequences from the same and other species and orthologous sequences from the same and other species. “Homology” refers to the level of similarity between two or more nucleic acid and/or amino acid sequences in terms of percent of positional identity, i.e., sequence similarity or identity. Homology also refers to the concept of similar functional properties among different nucleic acids or proteins. Thus, the compositions and methods of the invention further comprise homologues to the nucleotide sequences and polypeptide sequences of this invention. “Orthologous,” as used herein, refers to homologous nucleotide sequences and/or amino acid sequences in different species that arose from a common ancestral gene during speciation. A homologue of a nucleotide sequence of this invention has a substantial sequence identity, e.g., at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, and/or 100%, to said nucleotide sequence.

The term “overexpress,” “overexpresses”, “overexpression” or “upregulation” as used herein refers to higher levels of activity of a gene, e.g., transcription of the gene; higher levels of translation of mRNA into protein; and/or higher levels of production of a gene product, e.g., polypeptide, than would be in the cell in its native or control, e.g., not transformed with the particular heterologous or recombinant polypeptides being overexpressed, state. A typical example of an overexpressed gene is a gene under transcription control of another promoter as compared to the native promoter of the gene. Also, or alternatively, other changes in the control elements of a gene, such as enhancers, could be used to overexpress the particular gene. Furthermore, modifications that affect, i.e., increase, the translation of the mRNA transcribed from the gene could, alternatively or in addition, be used to achieve an overexpressed gene as used herein. These terms can also refer to an increase in the number of copies of a gene and/or an increase in the amount of mRNA and/or gene product in the cell. Overexpression can also be achieved by introducing one or more exogenous versions of the gene from another species. Overexpression can result in levels that are 25%, 50%, 100%, 200%, 500%, 1000%, 2000% or higher in the cell, or any range therein, as compared to control levels.

The term “downregulation” or “down-regulation” as used herein refers to lower levels of activity of a gene, e.g., transcription of the gene; lower levels of translation of mRNA into protein; and/or lower levels of production of a gene product, e.g., polypeptide, than would be in the cell in its native or control, e.g., not transformed with the particular heterologous or recombinant polypeptides being overexpressed, state. A typical example of downregulated gene is a gene under transcription control of another promoter with lower activity as compared to the native promoter of the gene. Also, or alternatively, other changes in the control elements of a gene, such as silencer elements, could be used to downregulate the particular gene. Furthermore, modifications that affect, i.e., decrease, the translation of the mRNA transcribed from the gene could, alternatively or in addition, be used to achieve a downregulated gene as used herein. These terms can also refer to a decrease in the amount of mRNA and/or gene product in the cell. In addition, this term can be used to refer to a gene that is disrupted or completely deleted. Downregulation can result in levels that are 10%, 20%, 50% or 100% lower in the cell, or any range therein, as compared to control levels.

As used herein, the terms “exogenous” or “heterologous” when used with respect to a nucleic acid (RNA or DNA), protein or gene refer to a nucleic acid, protein or gene which occurs non-naturally as part of the cell, organism, genome, RNA or DNA sequence into which it is introduced, including non-naturally occurring multiple copies of a naturally occurring nucleotide sequence. Such an exogenous gene could be a gene from another species or strain, a modified, mutated or evolved version of a gene naturally occurring in the host cell or a chimeric version of a gene naturally occurring in the host cell or fusion genes. In these former cases, the modification, mutation or evolution causes a change in the nucleotide sequence of the gene to thereby obtain a modified, mutated or evolved gene with another nucleotide sequence as compared to the gene naturally occurring in the host cell. Evolved gene refers to genes encoding evolved genes and obtained by genetic modification, such as mutation or exposure to an evolutionary pressure, to derive a new gene with a different nucleotide sequence as compared to the wild type or native gene. A chimeric gene is formed through the combination of portions of one or more coding sequences to produce a new gene. These modifications are distinct from a fusion gene, which merges whole gene sequences into a single reading frame and often retain their original functions.

An “endogenous”, “native” or “wild type” nucleic acid, nucleotide sequence, polypeptide or amino acid sequence refers to a naturally occurring or endogenous nucleic acid, nucleotide sequence, polypeptide or amino acid sequence. Thus, for example, a “wild type mRNA” is an mRNA that is naturally occurring in or endogenous to the organism. A “homologous” nucleic acid sequence is a nucleotide sequence naturally associated with a host cell into which it is introduced.

As used herein, the term “modified”, when it is used with respect to an organism, refers to a host organism that has been modified to increase production of fatty acids and/or derived products, as compared with an otherwise identical host organism that has not been so modified. In principle, such “modification” in accordance with the present disclosure may comprise any physiological, genetic, chemical, or other modification that appropriately alters production of fatty acids in a host organism as compared with such production in an otherwise identical organism which is not subject to the said modification. In most of the embodiments, however, the modification will comprise a genetic modification. In certain embodiments, as described herein, the modification comprises introducing genes into a host cell. In some embodiments, a modification comprises at least one physiological, chemical, genetic, or other modification; in other embodiments, a modification comprises more than one chemical, genetic, physiological, or other modification. In certain aspects where more than one modification is made use of, such modifications can include any combinations of physiological, genetic, chemical, or other modification (e.g., one or more genetic, chemical and/or physiological modification(s)). Genetic modifications which boost the activity of a polypeptide include, but are not limited to: introducing one or more copies of a gene encoding the polypeptide (which may distinguish from any gene already present in the host cell encoding a polypeptide having the same activity); altering a gene present in the cell to increase transcription or translation of the gene (e.g., altering, adding additional sequence to, replacement of one or more nucleotides, deleting sequence from, or swapping for example, regulatory, a promoter or other sequence); and altering the sequence (e.g., non-coding or coding) of a gene encoding the polypeptide to boost activity (e.g., by increasing enzyme activity, decrease feedback inhibition, targeting a specific subcellular location, boost mRNA stability, boost protein stability). Genetic modifications that reduce activity of a polypeptide include, but are not limited to: deleting a portion or all of a gene encoding the polypeptide; inserting a nucleic acid sequence which disrupts a gene encoding the polypeptide; changing a gene present in the cell to reduce transcription or translation of the gene or stability of the mRNA or polypeptide encoded by the gene (for example, by adding additional sequence to, altering, deleting sequence from, replacement of one or more nucleotides, or swapping for example, replacement of one or more nucleotides, a promoter, regulatory or other sequence).

The term “overproducing” is used herein in reference to the production of fatty acids or derived products in a host cell and indicates that the host cell is producing more fatty acids or derived products by virtue of the introduction of nucleic acid sequences which encode different polypeptides involved in the host cell's metabolic pathways or as a result of other modifications as compared with the unmodified host cell or wild-type cell.

As used herein, the term “flux”, “metabolic flux” or “carbon flux” refers to the rate of turnover of molecules through a given reaction or a set of reactions. Flux in a metabolic pathway is regulated by the enzymes involved in the pathway. Pathways or reactions characterized by a state of increased flux compared to a control have an increased rate of generation of products from given substrates. Pathways or reactions characterized by a state of decreased flux compared to a control have a decreased rate of generation of products from given substrates. Flux towards products of interest can be increased by removing or decreasing competitive reactions or by increasing the activities of enzymes involved in generation of said products.

As used herein, the term “acetyl-CoA derived products” refers to molecules for which acetyl-Coenzyme A (acetyl-CoA) is a precursor. Acetyl-CoA serves as a key precursor metabolite for the production of important cellular constituents such as fatty acids, sterols, and amino acids as well as it is used for acetylation of proteins. Besides these important functions it is also precursor metabolite for many other biomolecules, such as polyketides, isoprenoids, 3-hydroxypropionic acid, 1-butanol and polyhydroxyalkanoids, which encompass many industrially relevant chemicals.

As used herein the term “vector” is defined as a linear or circular DNA molecule comprising a polynucleotide encoding a polypeptide of the invention, and which is operably linked to additional nucleotides that ensure its expression.

“Introducing” in the context of a yeast cell means contacting a nucleic acid molecule with the cell in such a manner that the nucleic acid molecule gains access to the interior of the cell. Accordingly, polynucleotides and/or nucleic acid molecules can be introduced yeast cells in a single transformation event, in separate transformation events. Thus, the term “transformation” as used herein refers to the introduction of a heterologous nucleic acid into a cell. Transformation of a yeast cell can be stable or transient.

“Transient transformation” in the context of a polynucleotide means that a polynucleotide is introduced into the cell and does not integrate into the genome of the cell.

By “stably introducing” or “stably introduced” in the context of a polynucleotide introduced into a cell, it is intended that the introduced polynucleotide is stably incorporated into the genome of the cell, and thus the cell is stably transformed with the polynucleotide.

“Stable transformation” or “stably transformed” as used herein means that a nucleic acid molecule is introduced into a cell and integrates into the genome of the cell. As such, the integrated nucleic acid molecule is capable of being inherited by the progeny thereof, more particularly, by the progeny of multiple successive generations. Stable transformation as used herein can also refer to a nucleic acid molecule that is maintained extrachromasomally, for example, as a minichromosome.

Embodiments of the present invention also encompass variants of the polypeptides as defined herein. As used herein, a “variant” means a polypeptide in which the amino acid sequence differs from the base sequence from which it is derived in that one or more amino acids within the sequence are substituted for other amino acids. For example, a variant of SEQ ID NO:1 may have an amino acid sequence at least about 50% identical to SEQ ID NO:1, for example, at least about 55%, 60%, 65%, 70%, 75%, 80%, 85%, 90%, 91%, 92%, 93%, 94%, 95%, 96%, 97%, 98%, 99% or about 100% identical. The variants and/or fragments are functional variants/fragments in that the variant sequence has similar or identical functional enzyme activity characteristics to the enzyme having the non-variant amino acid sequence specified herein (and this is the meaning of the term “functional variant” as used throughout this specification).

A “functional variant” or “functional fragment” of any of the presented amino acid sequences, therefore, is any amino acid sequence which remains within the same enzyme category (i.e., has the same EC number) as the non-variant sequences. Methods of determining whether an enzyme falls within a particular category are well known to the skilled person, who can determine the enzyme category without use of inventive skill. Suitable methods may, for example, be obtained from the International Union of Biochemistry and Molecular Biology.

Amino acid substitutions may be regarded as “conservative” where an amino acid is replaced with a different amino acid with broadly similar properties. Non-conservative substitutions are where amino acids are replaced with amino acids of a different type.

By “conservative substitution” is meant the substitution of an amino acid by another amino acid of the same class, in which the classes are defined as follows:

### Class Amino Acid Examples

Nonpolar: A, V, L, I, P, M, F, W
Uncharged polar: G, S, T, C, Y, N, Q
Acidic: D, E
Basic: K, R, H.

As it is well known to those skilled in the art, altering the primary structure of a polypeptide by a conservative substitution may not significantly alter the activity of that polypeptide because the side-chain of the amino acid which is inserted into the sequence may be able to form similar bonds and contacts as the side chain of the amino acid which has been substituted out. This is so even when the substitution is in a region which is critical in determining the polypeptide's conformation.

In embodiments of the present invention, non-conservative substitutions are possible provided that these do not interrupt the enzyme activities of the polypeptides, as defined elsewhere herein. The substituted versions of the enzymes must retain characteristics such that they remain in the same enzyme class as the non-substituted enzyme, as determined using the NC-IUBMB nomenclature discussed above.

Broadly speaking, fewer non-conservative substitutions than conservative substitutions will be possible without altering the biological activity of the polypeptides. Determination of the effect of any substitution (and, indeed, of any amino acid deletion or insertion) is wholly within the routine capabilities of the skilled person, who can readily determine whether a variant polypeptide retains the enzyme activity according to aspects of the invention. For example, when determining whether a variant of the polypeptide falls within the scope of the invention (i.e., is a “functional variant or fragment” as defined above), the skilled person will determine whether the variant or fragment retains the substrate converting enzyme activity as defined with reference to the NC-IUBMB nomenclature mentioned elsewhere herein. All such variants are within the scope of the invention.

Using the standard genetic code, further nucleic acid sequences encoding the polypeptides may readily be conceived and manufactured by the skilled person, in addition to those disclosed herein. The nucleic acid sequence may be DNA or RNA, and where it is a DNA molecule, it may for example comprise a cDNA or genomic DNA. The nucleic acid may be contained within an expression vector, as described elsewhere herein.

Embodiments of the invention, therefore, encompass variant nucleic acid sequences encoding the polypeptides contemplated by embodiments of the invention. The term “variant” in relation to a nucleic acid sequence means any substitution of, variation of, modification of, replacement of, deletion of, or addition of one or more nucleotide(s) from or to a polynucleotide sequence, providing the resultant polypeptide sequence encoded by the polynucleotide exhibits at least the same or similar enzymatic properties as the polypeptide encoded by the basic sequence. The term includes allelic variants and also includes a polynucleotide (a “probe sequence”) which substantially hybridizes to the polynucleotide sequence of embodiments of the present invention. Such hybridization may occur at or between low and high stringency conditions. In general terms, low stringency conditions can be defined as hybridization in which the washing step takes place in a 0.330-0.825 M NaCl buffer solution at a temperature of about 40-48° C. below the calculated or actual melting temperature (Tm) of the probe sequence (for example, about ambient laboratory temperature to about 55° C.), while high stringency conditions involve a wash in a 0.0165-0.0330 M NaCl buffer solution at a temperature of about 5-10° C. below the calculated or actual Tm of the probe sequence (for example, about 65° C.). The buffer solution may, for example, be SSC buffer (0.15M NaCl and 0.015M tri-sodium citrate), with the low stringency wash taking place in 3×SSC buffer and the high stringency wash taking place in 0.1×SSC buffer. Steps involved in hybridization of nucleic acid sequences have been described for example in Molecular Cloning, a laboratory manual [second edition] Sambrook et al. Cold Spring Harbor Laboratory, 1989, for example in Section 11 “Synthetic Oligonucleotide Probes” thereof (herein incorporated by reference)

Preferably, nucleic acid sequence variants have about 55% or more of the nucleotides in common with the nucleic acid sequence of embodiments of the present invention, more preferably at least 60%, 65%, 70%, 80%, 85%, or even 90%, 95%, 98% or 99% or greater sequence identity.

Variant nucleic acids of the invention may be codon-optimized for expression in a particular host cell.

As used herein, “sequence identity” refers to sequence similarity between two nucleotide sequences or two peptide or protein sequences. The similarity is determined by sequence alignment to determine the structural and/or functional relationships between the sequences.

Sequence identity between amino acid sequences can be determined by comparing an alignment of the sequences using the Needleman-Wunsch Global Sequence Alignment Tool available from the National Center for Biotechnology Information (NCBI), Bethesda, Md., USA, for example via http://blast.ncbi.nlm.nih.gov/Blast.cgi, using default parameter settings (for protein alignment, Gap costs Existence:11 Extension:1). Sequence comparisons and percentage identities mentioned in this specification have been determined using this software.

An aspect of the embodiments relates to a fungal cell suitable for the production of fatty acids and/or fatty acid-derived products. The fungal cell is, in this aspect, genetically modified for overexpression of an acetyl-CoA carboxylase (EC 6.4.1.2) and a pyruvate carboxylase (EC 6.4.1.1).

In the following, various embodiments of the present invention will be described in more detail.

In an embodiment, the fungal cell is a fungal cell selected from a group consisting of Saccharomyces, Kluyveromyces, Zygosaccharomyces, Candida, Hansenula, Tonulopsis, Kloeckera, Pichia, Schizosaccharomyces, Trigonopsis, Brettanomyces, Debaromyces, Nadsonia, Lipomyces, Cryptococcus, Aureobasidium, Trichosporon, Lipomyces, Rhodotorula, Yarrowia, Rhodosporidium, Phaffia, Schwanniomyces, Aspergillus and Ashbya. In a particular embodiment, the fungal cell can be Saccharomyces cerevisiae, Pichia pastoris, Ashbya gossypii, Saccharomyces boulardii, Zygosaccharomyces bailii, Kluyveromyces lactis, Rhodosporidium toruloides and Yarrowia lipolytica. Saccharomyces cerevisiae and Yarrowia lipolytica are preferred yeast species.

According to the invention, the fungal cell is engineered for increased supply of acetyl-CoA and/or malonyl-CoA, precursors for fatty acids. This is achieved by upregulation of genes coding for pyruvate carboxylase (PYC) to increase oxaloacetate supply and acetyl-CoA carboxylase (ACC) to increase pull towards malonyl-CoA. For instance, production of the enzymes PYC1 (YGL062W) and ACC1 (YNR016C) could be upregulated in the fungal cell.

In an embodiment, the fungal cell is genetically modified for overexpression of a mitochondrial pyruvate carrier to increase pyruvate import into mitochondria under high glucose. For instance, production of the proteins MPC1 (YGL080W) and/or MPC3 (YGR243W) could be upregulated in the fungal cell. These modifications could be combined with the modifications above, or be completely independent.

In an embodiment, the fungal cell is further genetically modified for overexpression of a citrate and oxoglutarate carrier protein, which is an antiporter contributing to increased NADPH in the cytosol. For instance, production of the protein YHM2 (YMR241W) could be upregulated in the fungal cell.

In an embodiment, the fungal cell is further genetically modified for overexpression of a cytosolic isocitrate dehydrogenase (IDH) (EC 1.1.1.42). For instance, production of the enzyme IDP2 (YLR174W) could be upregulated in the fungal cell.

Any or all of the above mentioned embodiments could be combined in the fungal cell.

According to the invention, the fungal cell is genetically modified for enhanced activity of acetyl-CoA carboxylase, preferably ACC1 (SEQ ID NO: 1), or a variant of SEQ ID NO: 1. This may be achieved via overexpression of ACC1 and/or via expression or overexpression of a mutant ACC1 variant with higher activity. Illustrative, but non-limiting, example of such mutant ACC1 variants include ACC1 from Saccharomyces cerevisiae, in which serine 659 in SEQ ID NO: 1 and/or serine 1157 in SEQ ID NO: 1 is/are replaced with alanine.

According to the invention, the fungal cell is genetically modified for enhanced activity of pyruvate carboxylase, preferably PYC1 (SEQ ID NO: 2), or a variant of SEQ ID NO: 2. This can be achieved, for example, via overexpression of PYC1.

Another aspect of the embodiments relates to a fungal cell for the production of fatty acids and/or fatty acid-derived products. The fungal cell is genetically modified for overexpression of an acetyl-CoA carboxylase, preferably ACC1, and for overexpression of a mitochondrial pyruvate carrier, preferably MPC1 and/or MPC3. In another embodiment, the fungal cell is genetically modified for overexpression of a pyruvate carboxylase, preferably PYC1, and for overexpression of a mitochondrial pyruvate carrier, preferably MPC1 and/or MPC3.

In an embodiment, the fungal cell is genetically modified for:
Overexpression or enhanced activity of an acetyl-CoA carboxylase, preferably ACC1, and
Overexpression or enhanced activity of a pyruvate carboxylase, preferably PYC1, and
Overexpression or enhanced activity of a mitochondrial pyruvate carrier, preferably MPC1 and/or MPC3

In an embodiment, the fungal cell also comprises modifications focusing on increasing the synthesis of citrate as precursor for acetyl-CoA in the fungal cell. This could be achieved by overexpression of a citrate synthase (EC 2.3.3.16), such as overexpression of S. cerevisiae citrate synthase ScCIT1 (YNR001C) and/or overexpression of citrate synthase RtCIT1 from Rhodosporidium toruloides (SEQ ID NO: 3), or a variant of SEQ ID NO: 3.

In another embodiment, the fungal cell instead or in addition comprises modifications to enhance mitochondrial oxaloacetate production required for citrate synthesis by targeting the cytosolic pyruvate carboxylase into the mitochondria (mPYC1) (SEQ ID NO: 4), or a variant of SEQ ID NO: 4.

In another embodiment, the fungal cell instead or in addition comprises modifications to enhance the flux from citrate to acetyl-CoA via expression, preferably overexpression, of an ATP-citrate-lyase (EC 2.3.3.8), preferably a heterologous ATP-citrate lyase, such as AnACL from Aspergillus nidulans (SEQ ID NO: 5), or a variant of SEQ ID NO: 5.

In another embodiment, the fungal cell instead or in addition comprises modifications to enhance export of citrate from the mitochondria to the cytosol by downregulation of the gene coding for the mitochondrial NAD+-dependent isocitrate dehydrogenase IDH2 (YOR136W, EC 1.1.1.41) (SEQ ID NO: 14).

This modification could be combined with any of the modifications above or be completely independent. In the latter case, the invention relates to a fungal cell, preferably a fungal cell for the production of fatty acids and/or fatty acid-derived products, wherein the fungal cell is genetically modified for downregulation of the gene coding for the mitochondrial NAD+-dependent isocitrate dehydrogenase IDH2 (YOR136W, EC 1.1.1.41) (SEQ ID NO: 14).

The downregulation of the endogenous mitochondrial NAD+-dependent isocitrate dehydrogenase can be achieved by having a native promoter of the mitochondrial NAD+-dependent isocitrate dehydrogenase replaced by a comparatively weaker promoter. For instance, the native promoter can be replaced by a weaker promoter selected from the group consisting of pINH1, pSDH4, pATP5, pGSY2, pGSP2, and pRBK1.

Any of the above described modifications can be combined.

A further aspect of the embodiments relates to a fungal cell for the production of fatty acids and/or fatty acid-derived products. The fungal cell is genetically modified for overproduction of an acetyl-CoA carboxylase, preferably ACC1, and for overexpression of a citrate synthase, preferably CIT1 from Rhodosporidium toruloides. In another embodiment, the fungal cell is genetically modified for overexpression of a citrate synthase, preferably CIT1 from Rhodosporidium toruloides, and for overexpression of a pyruvate carboxylase, preferably PYC1.

In an embodiment, the fungal cell is genetically modified for:
Overexpression or enhanced activity of an acetyl-CoA carboxylase, preferably ACC1, and
Overexpression or enhanced activity of a pyruvate carboxylase, preferably PYC1, and
Overexpression or enhanced activity of a citrate synthase, preferably CIT1 from Rhodosporidium toruloides.

In an embodiment, the fungal cell is genetically modified for overexpression of the citrate synthase, preferably CIT1 from Rhodosporidium toruloides, and for overexpression of a citrate and oxoglutarate carrier protein, preferably YHM2.

In an embodiment, the fungal cell is instead or in addition genetically modified for overexpression of the citrate synthase, preferably CIT1 from Rhodosporidium toruloides, and for overexpression of a cytosolic isocitrate dehydrogenase, preferably IDP2.

In an embodiment, the fungal cell is modified for overexpression of the citrate synthase, preferably CIT1 from Rhodosporidium toruloides, and for overexpression of an ATP-citrate-lyase, preferably AnACL from Aspergillus nidulans.

In an embodiment, the fungal cell with any of the above modifications is further genetically modified for downregulation of an endogenous mitochondrial NAD+-dependent isocitrate dehydrogenase, for example IDH2. Downregulation of the mitochondrial NAD+-dependent isocitrate dehydrogenase can be accomplished by replacement of the native promoter of the mitochondrial NAD+-dependent isocitrate dehydrogenase with a weaker promoter, preferably taken from the group consisting of pINH1, pSDH4, pATP5, pGSY2, pGSP2, pRBK1.

In a preferred embodiment, a fungal cell is modified for increased NADPH supply for elongation and reduction reactions. This could be achieved by downregulation of a gene encoding for an endogenous phosphoglucose isomerase (PGI) (YBR196C, EC 5.3.1.9), thereby directing the metabolic flux into the pentose phosphate pathway (PPP) for increased generation of NADPH. Thus, in an embodiment, the fungal cell with any of the above modifications is further genetically modified for downregulation of the endogenous phosphoglucose isomerase, for example PGI1. Downregulation of the endogenous phosphoglucose isomerase may be accomplished by replacement of the native promoter controlling the expression of the phosphoglucose isomerase by a weaker promoter, preferably selected from the group consisting of pISU1, pATP14, pQCR10, pCOX9, pNAT1 and pHXT1. This modification could be combined with any of the modifications above or be completely independent. In the latter case, the invention relates to a fungal cell, preferably a fungal cell for the production of fatty acids and/or fatty acid-derived products, wherein the fungal cell is genetically modified for downregulation of the endogenous phosphoglucose isomerase, for example PGI1 (YBR196C, EC 5.3.1.9).

In another preferred embodiment, NADPH supply in the fungal cell is further increased by overexpressing genes coding for a glucose-6-phosphate dehydrogenase (ZWF1; YNL241C, EC 1.1.1.49) catalyzing the irreversible and rate limiting first step of PPP and is responsible for the main NADPH regeneration from NADP+; GND1 (YHR183W, EC 1.1.1.44) encoding the major phosphogluconate dehydrogenase that catalyzes the second oxidative reduction of NADP+ to NADPH; TKL1 (YPR074C, EC 2.2.1.1) and TAL1 (YLR354C, EC 2.2.1.2) encoding a transketolase and a transaldolase of the non-oxidative PPP. Other ways to increase NADPH could include expression of a gene coding for a non-phosphorylating NADP+-dependent glyceraldehydes-3-phosphate dehydrogenase (GAPN; preferably from Streptococcus mutans), or a phosphoketolase pathway e.g., from Aspergillus nidulans (heterologous expression of xpkA and ack). In an embodiment, the fungal cell is a Saccharomyces cerevisiae cell genetically modified for overexpression of an endogenous GDH2 (YDL215C, EC 1.4.1.2) gene encoding a NAD-dependent glutamate dehydrogenase. These modifications could be combined with any of the modifications above, or be completely independent.

In a preferred embodiment, the fungal cell is genetically modified for overexpression of an acetyl-CoA carboxylase, preferably ACC1, and overexpression of a pyruvate carboxylase, preferably PYC1, and any of the following:
Overexpression of a mitochondrial pyruvate carrier, preferably MPC1 and/or MPC3, and/or
Overexpression of a citrate synthase, preferably CIT1 from Rhodosporidium toruloides, and/or
Overexpression of a citrate and oxoglutarate carrier protein, preferably YHM2, and/or
Overexpression of a cytosolic isocitrate dehydrogenase, preferably IDP2, and/or
Overexpression of an ATP-citrate lyase, preferably from Aspergillus nidulans, and/or
Downregulation of the endogenous mitochondrial NAD+-dependent isocitrate dehydrogenase, preferably IDH2, and/or
Downregulation of the endogenous phosphoglucose isomerase, preferably PGI1.

In an embodiment, the fungal cell is a S. cerevisiae with the following modifications:
Upregulation of genes coding for PYC1 (YGL062W, EC 6.4.1.1) (increasing oxaloacetate supply), ACC1 (YNR016C; EC 6.4.1.2) (increasing pull towards malonyl-CoA), IDP2 (YLR174W, EC 1.1.1.42), YHM2 (YMR241W) (antiporter contributing to increased NADPH in cytosol), MPC1 (YGL080W) and MPC3 (YGR243W) (increase pyruvate import into mitochondria under high glucose), and
Downregulation of endogenous genes encoding for a phosphoglucose isomerase PGI1 (YBR196C, EC 5.3.1.9), thereby directing the metabolic flux into the pentose phosphate pathway for increased generation of NADPH;
Increasing NADPH supply in the fungal cell by overexpressing genes coding for a glucose-6-phosphate dehydrogenase (ZWF1, YNL241C, EC 1.1.1.49), the major phosphogluconate dehydrogenase GND1 coded by GND1 (YHR183W, EC 1.1.1.44), a transketolase and a transaldolase coded by TKL1 (YPR074C, EC 2.2.1.1) and TAL1 (YLR354C, EC 2.2.1.2); and
Enhanced export of citrate from the mitochondria to the cytosol by downregulation of the gene coding for the mitochondrial NAD+-dependent isocitrate dehydrogenases IDH2 (YOR136W, EC 1.1.1.41) and IDP1 (YDL066W, EC 1.1.1.42) (SEQ ID NO: 6), or a variant of SEQ ID NO: 6.

In an embodiment, the fatty acid production in the fungal cell could be increased by redirecting the flux from cell growth to fatty acid production by limiting cell growth through downregulation of essential genes by replacing a native promoter of the essential gene by a carbon-source dependent promoter. For example, the P_{HXT1} promoter could be introduced to control the expression of the essential genes ERG9 (YHR190W, EC 2.5.1.21) and/or LEU2 (YCL018W, EC 1.1.1.85) in, for instance, S. cerevisiae or Y. lipolytica to limit cell growth at low glucose concentrations. These modifications could be combined with any of the modifications above, or be completely independent.

In an embodiment, the fatty acid production in the fungal cell factory could be increased by redirecting the flux from cell growth to fatty acid production by limiting cell growth through limiting supply of an essential nutrient. For example the supply of nitrogen is limiting cell growth in S. cerevisiae and leads to increase of fatty acid production.

In an embodiment, any of the modifications above may be combined with genetic modifications in the fungal cell to abolish ethanol formation. This includes downregulating pyruvate decarboxylase activity in the fungal cell by deletion of one or more genes coding for pyruvate decarboxylases catalyzing the decarboxylation of pyruvate to acetaldehyde. For example, deletion of the genes PDC1, PDC5, and/or PDC6 (YLR044C, YLR134W, YGR087C, SEQ ID NO: 7-9), or variants of SEQ ID NI: 7-9, in the yeast S. cerevisiae leads to abolishment of ethanol formation (Zhang et al., 2015.) and all or some of these could be deleted or downregulated to decrease or completely abolish ethanol production.

In another embodiment, any of the modifications above are combined with genetic modifications to restore growth on glucose of fungal cells abolished for ethanol formation. This includes inserting specific mutations in the gene MTH1^{81D} (YDR277C) or truncated versions of the MTH1 gene (SEQ ID NO: 10), coding for a version with higher activity.

In another embodiment any of the modifications above are combined with adaptive laboratory evolution (ALE) to restore growth on glucose of fungal cells abolished for ethanol formation. This includes e.g., exposing the engineered yeast cell stepwise to lower concentrations of ethanol with at the same time increasing the concentration of glucose in the cultivation medium.

In an embodiment, the fungal cell is modified to abolish ethanol formation as well as lower the activity of a fructose-1,6-bisphosphate (FBP)-sensitive pyruvate kinase, for example PYK1 (SEQ ID NO: 11), also known as CDC19, or a variant of SEQ ID NO: 11. In another embodiment, the fungal cell is alternatively, or in addition, modified for increased activity of a FBP-insensitive pyruvate kinase, such as PYK2 (SEQ ID NO: 12), or a variant of SEQ ID NO: 12. Downregulation of PYK1 activity can be accomplished by deletion, promoter replacement, or mutation, for example in the R68, K196, or R91 residues. Increased activity of PYK2 can be accomplished by overexpression, for example via promoter replacement or introduction of additional copies. For example, in a preferred embodiment a fungal cell with deletions in the genes PDC1 and PDC5 is further modified for downregulation of PYK1 and overexpression of PYK2. These modifications can be combined with the above modifications for fatty acid production, or be completely independent.

In an embodiment, the fungal cell is genetically modified for:
Overexpression or enhanced activity of an acetyl-CoA carboxylase, preferably by overexpression of ACC1, and
Overexpression or enhanced activity of a pyruvate carboxylase, preferably by overexpression of PYC1, and
Downregulation or decreased activity of a pyruvate decarboxylase, preferably by deletion of PDC1, PDC5 and/or PDC6, and
Downregulation or decreased activity of a FBP-sensitive pyruvate kinase, preferably by deletion or downregulation of PYK1.

In an embodiment, the fungal cell is genetically modified for:
Overexpression or enhanced activity of an acetyl-CoA carboxylase, preferably by overexpression of ACC1, and
Overexpression or enhanced activity of a pyruvate carboxylase, preferably by overexpression of PYC1, and
Downregulation or decreased activity of a pyruvate decarboxylase, preferably by deletion of PDC1, PDC5 and/or PDC6, and
Overexpression or enhanced activity of a FBP-insensitive pyruvate kinase, preferably by overexpression of PYK2.

In an embodiment, the fungal cell is a Saccharomyces cerevisiae yeast cell genetically modified for overproduction of an acetyl-CoA-derived product. The yeast cell is further modified for decreased ethanol production via deletion or downregulation of PDC1, PDC5 and/or PDC6. In a further embodiment the yeast cell is genetically modified for deletion or downregulation of PYK1. In a further embodiment the aforementioned yeast cell is further modified for overexpression of PYK2. These modifications can be combined with the above modifications for fatty acid production, or be completely independent.

In a preferred embodiment, the fungal cell is genetically modified for:
Overexpression or enhanced activity of an acetyl-CoA carboxylase, preferably by overexpression of ACC1, and
Overexpression or enhanced activity of a pyruvate carboxylase, preferably by overexpression of PYC1, and
Downregulation or decreased activity of a pyruvate decarboxylase, preferably by deletion of PDC1, PDC5 and/or PDC6, and
Downregulation or decreased activity of a FBP-sensitive pyruvate kinase, preferably by deletion or downregulation of PYK1, and
Overexpression or enhanced activity of a FBP-insensitive pyruvate kinase, preferably by overexpression of PYK2.

In some embodiments, the fungal cell has increased production capacities for free fatty acids incorporating genetic modifications, including deletion of the genes coding for a fatty aldehyde dehydrogenase (HFD1, YMR110C, EC 1.2.1.3), a fatty-acyl coenzyme A oxidase (POX1, YGL205W, EC 1.3.3.6) and/or fatty acyl-CoA synthetases FAA1 (YOR317W, EC 6.2.1.3) and FAA4 (YMR246W, EC 6.2.1.3). In addition, or alternatively, the fungal cell is genetically modified for overexpression of heterologous genes including Mus musculus ATP-citrate-lyase (MmACL, EC 2.3.3.8), R. toruloides malic enzymes (RtME, EC 1.1.1.40), a truncated version of E. coli thioesterase (tesA, EC 3.1.2.2.) and overexpression of R. toruloides FAS encoding genes (RtFAS1 and RtFAS2). In an embodiment, to further increase free fatty acid production endogenous genes coding for mitochondrial citrate transporter (CTP1, YBR291C) and malate dehydrogenase (MDH3, YDL078C, EC 1.1.1.37) may be overexpressed. These modifications could be combined with any of the modifications above, or be completely independent.

In an embodiment, the fungal cell is genetically modified for:
Deletion in POX1, FAA1 and FAA4, and
Overexpression of a thioesterase, preferably tesA from Escherichia coli, and
Overexpression of endogenous ACC1 and PYC1, and
Deletion of PDC1, PDC5 or PDC6, and
Deletion of PYK1, and
Overexpression of PYK2.

In another embodiment, the fungal cell is Saccharomyces cerevisiae and is genetically modified for:
Deletion of HFD1, POX1, FAA1 and FAA4, and
Overexpression of an ATP-citrate lyase, preferably from Mus Musculus or Aspergillus nidullans, and
Overexpression of a cytosolic NADP+-dependent malic enzyme, preferably from Rhodosporidium toruloides, and
Overexpression of endogenous CTP1 and MDH3, and
Overexpression of a thioesterase, preferably tesA from Escherichia coli, and
Overexpression of FAS1 and/or FAS2, preferably from Rhodosporidium toruloides, and
Overexpression of endogenous ACC and PYC1, and
Overexpression of CIT1, preferably from Rhodosporidium toruloides, and
Overexpression of endogenous PDA1, IDP2 and YHM2, and
Downregulation of PGI1, and
Overexpression of endogenous GND1, TKL1, TAL1, and ZWF1, and
Downregulation of IDH2, and
Deletion of PDC1, PDC5 and PDC6, and
Deletion of PYK1, and
Overexpression of PYK2.

In an embodiment, increased production of fatty acids and/or fatty acid-derived products is, instead or in addition, achieved through overexpression of one or more endogenous yeast genes selected from the group consisting of M-Phase Phosphoprotein 6 homolog (MPP6) (SEQ ID NO: 27), or a variant of SEQ ID NO:27; Acyl Carrier Protein (ACP1) (SEQ ID NO: 28), or a variant of SEQ ID NO: 28; EthanolaminePhosphoTransferase (EPT1) (SEQ ID NO: 29), or a variant of SEQ ID NO: 29; Long chain fatty acyl-CoA synthetase (FAA1) (SEQ ID NO: 30), or a variant of SEQ ID NO: 30; Mitochondrial phosphatidylglycerophosphatase (GEP4) (SEQ ID NO: 31), or a variant of SEQ ID NO: 31; ADP-ribosylation factor-binding protein GGA2 (GGA2) (SEQ ID NO: 13), or a variant of SEQ ID NO: 13; NADP-dependent isocitrate dehydrogenase (IDP3) (SEQ ID NO: 32), or a variant of SEQ ID NO: 32; Phosphatidylinositol 4,5-bisphosphate 5-phosphatase (INP54) (SEQ ID NO: 33), or a variant of SEQ ID NO: 33; Lipid phosphate phosphatase (LPP1) (SEQ ID NO: 34), or a variant of SEQ ID NO: 34; Mitochondrial NADH-cytochrome b5 reductase (MCR1) (SEQ ID NO: 35), or a variant of SEQ ID NO: 35; sphingolipid homeostasis protein ORM1 (ORM1) (SEQ ID NO: 36), or a variant of SEQ ID NO: 36; Restriction of telomere capping protein 3 (RTC3) (SEQ ID NO: 37), or a variant of SEQ ID NO: 37; SPO7 (SEQ ID NO: 38), or a variant of SEQ ID NO: 38; TriGlyceride Lipase (TGL1) (SEQ ID NO: 39), or a variant of SEQ ID NO: 39; YFT2 (SEQ ID NO: 40), or a variant of SEQ ID NO: 40. These modifications can be combined with any of the above modifications or be completely independent. In a preferred embodiment, the fungal cell overexpresses GGA2 (SEQ ID NO: 13), or a variant of SEQ ID NO: 13. This modification can be combined with any of the above modifications or be completely independent.

In an embodiment, the fungal cell overexpresses GGA2 and INP54. This modification can be combined with any of the above modifications or be completely independent.

In an embodiment, the fungal cell overexpresses GGA2 and IDP3. This modification can be combined with any of the above modifications or be completely independent.

In an embodiment, the fungal cell overexpresses GGA2 and TGL1. This modification can be combined with any of the above modifications or be completely independent.

In an embodiment, the fungal cell overexpresses INP54 and IDP3. This modification can be combined with any of the above modifications or be completely independent.

In an embodiment, the fungal cell overexpresses GGA2, INP54 and IDP3. This modification can be combined with any of the above modifications or be completely independent.

In an embodiment, the fungal cell overexpresses GGA2, INP54, IDP3 and TGL1. This modification can be combined with any of the above modifications or be completely independent.

In an embodiment, the fungal cell overexpresses GGA2 and is used for production of a fatty acid composed of 16 carbons, including, but not limited to, palmitic acid or palmitoleic acid. This modification can be combined with any of the above modifications or be completely independent.

In an embodiment, the fungal cell overexpresses EPT1 and is used for production of a fatty acid composed of 16 carbons, including, but not limited to, palmitic acid or palmitoleic acid. This modification can be combined with any of the above modifications or be completely independent.

In an embodiment, the fungal cell overexpresses IDP3 and is used for production of a fatty acid composed of 16 carbons, including, but not limited to, palmitic acid or palmitoleic acid. This modification can be combined with any of the above modifications or be completely independent.

In an embodiment, the fungal cell overexpresses TGL1 and is used for production of a fatty acid composed of 16 carbons, including, but not limited to, palmitic acid or palmitoleic acid. This modification can be combined with any of the above modifications or be completely independent.

In an embodiment, the fungal cell overexpresses RTC3 and is used for production of oleic acid. This modification can be combined with any of the above modifications or be completely independent.

In an embodiment, the fungal cell for production of fatty acids is genetically modified for:
Overexpression or enhanced activity of an acetyl-CoA carboxylase and/or a pyruvate carboxylase, and
Overexpression or enhanced activity of GGA2

In an embodiment, the fungal cell for production of fatty acids is genetically modified for:
Overexpression or enhanced activity of an acetyl-CoA carboxylase and/or a pyruvate carboxylase, and
Overexpression or enhanced activity of a citrate synthase and/or a mitochondrial pyruvate carrier, and
Overexpression or enhanced activity of GGA2.

In an embodiment, the fungal cell is genetically modified for:
Reduction in ethanol formation by downregulation or deletion of genes selected from the group consisting of PDC1, PDC5 and PDC6, and
Downregulation of PYK1 and/or overexpression of PYK2, and
Overexpression of GGA2.

In some embodiments, the fungal cell is further modified for reduced expression or knockout of pathways competing for fatty acids. This can include genes selected from a group consisting of acyl-CoA: sterol acyltransferase (ARE1, YCR048W, EC 2.3.1.26; ARE2, YNR019W, EC 2.3.1.26), diacylglycerol acyltransferase (DGA1, YOR245C, EC 2.3.1.20), lecithin cholesterol acyl transferase (LR01, YNR008W, EC 2.3.1.158), fatty-acyl coenzyme A oxidase (POX1, YGL205W, EC 1.3.3.6).

In some embodiments, the fungal cell is further modified for reduced expression or knockout of genes involved in fatty acid activation in order to increase accumulation of free fatty acids. This can include, for example, fatty acyl-coA synthetases, such as the genes FAA1 (YOR317W, EC 6.2.1.3), FAA2 (YER015W, EC 6.2.1.3), FAA3 (YIL009W, EC 6.2.1.3), FAA4 (YMR246W, EC 6.2.1.3) and FAT1 YBR041W, EC 6.2.1.3).

In some embodiments, endogenous fatty acid genes are de-regulated. For example, the elongation genes, ELO1, ELO2 and/or ELO3, can be de-regulated if shorter-chain (less than 16 carbons) or longer-chain (more than 18 carbons) fatty acids are required so that a) their expression and/or activity is lower during the production phase than during the growth phase and (b) the expression and/or activity during the production phase is lower than the endogenous expression and/or activity during this phase as compared to a non-de-regulated control. Such de-regulation could be achieved via promoter replacement or via other means as described above.

In some embodiments, the fungal cell is modified for increased conversion of fatty acyl CoAs to free fatty acids with the overexpression of a thioesterase. This can be done via overexpression of endogenous thioesterases, or heterologous thioesterases, such as mammalian ACOT genes, for instance, Homo sapiens ACOT2 (GenBank: P 006812.3), Homo sapiens ACOT9 (Genbank: P_001028755.2), Rattus norvegicus ACOT2 (GenBank: P_620262.2) or Rattus norvegicus ACOT 1 (Genbank: P_112605.1).

In some embodiments, any of the modifications above can be combined with expression of acyl-CoA oxidases (EC 1.3.3.6) or acyl-CoA dehydrogenases (EC 1.3.8.7) to facilitate chain shortening of the fatty acid.

In an embodiment, the fungal cell is genetically modified for overexpression of at least one enzyme involved in fatty acid synthesis in the fungal cell and selected from the group consisting of a fatty acid synthase, such as FAS1 and/or FAS2; an acyl-CoA-binding protein, such as ACB1; a mitochondrial citrate transporter, such as CTP1; a malate dehydrogenase, such as MDH3; cytosolic isocitrate dehydrogenase, such as IDP2; a citrate and oxoglutarate carrier protein, such as YHM2; a mitochondrial pyruvate carrier, such as MPC1 and/or MPC3; a citrate synthase, such as CIT1; a glucose-6-phosphate dehydrogenase, such as ZWF1; a transketolase, such as TKL1; a transaldolase, such as TAL1; and a glutamate dehydrogenase, such as GDH2.

In an embodiment, the fungal cell is genetically modified for attenuated activity or downregulation of at least one enzyme involved in fatty acid biosynthesis in the fungal cell and selected from the group consisting of a fatty-acyl-CoA synthetase, such as FAA1, FAA2, FAA3, FAA4 and/or FAT1; a fatty aldehyde dehydrogenase, such as HFD1; a fatty-acyl-CoA oxidase, such as POX1; a mitochondrial isocitrate dehydrogenase, such as IDH2 and/or IDP1; a phosphoglucose isomerase, such as PGI1; an acyl-CoA-sterol acyltransferase, such as ARE1 and/or ARE2; a diacylglycerol acyltransferase, such as DGA1; and a lecithin cholesterol acyl transferase, such as LRO1.

In an embodiment, the fungal cell is a yeast cell.

In an embodiment, the fatty acid is selected from the group consisting of stearic acid, oleic acid, palmitic acid, palmitoleic acid and a mixture thereof.

In some embodiments, the fatty acids are further processed into fatty acid-derived products, such as fatty alcohols, fatty aldehydes, fatty esters, hydrocarbons, triacylglycerides, lactones, phospholipids, etc. Preferably hydroxy fatty acids, fatty alcohols or fatty aldehydes.

This can be achieved by introduction of additional genes encoding the appropriate fatty acid modification activity. For example, conversion of fatty acids to fatty alcohols can be facilitated by expression of a fatty acyl-CoA reductase (FAR; EC 1.2.1.84). Conversion of fatty acids to hydroxy fatty acids can be achieved by expression of a fatty acid hydratase (EC 4.2.1.53) or a fatty acid hydroxylase (EC 1.11.2.4, EC 1.14.14.1, EC 1.14.15.12 or EC 1.14.15.3). Conversion of fatty acids to branched-chain fatty acids can be achieved by expression of a fatty acid methyltransferase. Conversion of fatty acids to epoxy fatty acids can be achieved by expression of a peroxygenase (EC 1.11.2.3). Conversion of free fatty acids to fatty aldehydes can be achieved by expression of carboxylic acid reductase (CAR; EC 1.2.99.6), while conversion of fatty acyl-CoAs to fatty aldehydes can be achieved by expression of an aldehyde-forming fatty acyl-CoA reductase (EC 1.2.1.50).

In some embodiments the fungal cell is genetically modified to increase the production of unsaturated fatty acids. This can be achieved by overexpressing a desaturase, for example, a fatty acyl-CoA desaturase. Examples of desaturases can include: a delta 3 desaturase, delta 4 desaturase, delta 5 desaturase, delta 6 desaturase, delta 7 desaturase, delta 8 desaturase, delta 9 desaturase, delta 10 desaturase, delta 11 desaturase, delta 12 desaturase, delta 13 desaturase, delta 14 desaturase, delta 15 desaturase, delta 16 desaturase and delta 17 desaturase. In some embodiments the desaturase might have a bifunctional or trifunctional activity with any combination of the above.

In some embodiments, a fungal cell comprises of overexpression of at least one exogenous or endogenous gene encoding a transport protein to facilitate increased secretion of fatty acids or fatty acid derived products into the media. The transport protein can be selected from the group consisting of an ATP-binding cassette (ABC) protein, a lipid transfer protein (LTP), a fatty acid transporter protein (FATP) and a plant wax ester transporter, preferably selected from the group consisting of ABCG11, ABCG12, LTPG1 and/or LTPG2. For example, ABC transporters of Arabidopsis such as ABCG11 and/or ABCG12 as well as lipid transfer proteins (LTPs) such as LTPG1 and LTPG2 can be introduced into a host cell. In some embodiments, fatty acid transporter (FATP) genes from species including Saccharomyces, Drosophila, Mycobacteria, or mammalian species can be introduced into a host cell. In some embodiments, the transporter protein increases the amount of fatty acids or fatty acid derived products such as fatty alcohols, fatty aldehydes, fatty esters, hydrocarbons, triacylglycerides, hydroxy fatty acids, dicarboxylic fatty acids, branched chain fatty acids, epoxy fatty acids or lactones, released into the growth media of a microorganism. Preferred transport proteins include FATP1 from Homo sapiens (Genbank: NP_940982; XP_352252), FATP4 from Homo sapiens (Genbank; NP_005085), and FAT1 from S. cerevisiae (Genebank: NP_009597). Expression of a transporter protein can in some embodiments also increase production of fatty acids or fatty acid derived products by a host strain. In a preferred embodiment, expression of FATP1 from Homo sapiens (Genbank: NP_940982) or another mammalian source in S. cerevisiae or Y. lipolytica facilitates the export into the growth medium of fatty acids or fatty acid derived products such as fatty alcohols, fatty aldehydes, fatty esters, hydrocarbons, triacylglycerides, hydroxy fatty acids, dicarboxylic fatty acids, branched chain fatty acids, epoxy fatty acids or lactones. In another preferred embodiment, expression of FATP4 from Homo sapiens (Genbank; NP_005085) or another mammalian source in S. cerevisiae or Y. lipolytica facilitates the export into the growth medium of fatty acids or fatty acid derived products such as fatty alcohols, fatty aldehydes, fatty esters, hydrocarbons, triacylglycerides, hydroxy fatty acids, dicarboxylic fatty acids, branched chain fatty acids, epoxy fatty acids or lactones. In yet another preferred embodiment overexpression of FAT1 from S. cerevisiae Genebank: NP_009597) in S. cerevisiae or Y. lipolytica facilitates the export into the growth medium of fatty acids or fatty acid derived products such as fatty alcohols, fatty aldehydes, fatty esters, hydrocarbons, triacylglycerides, hydroxy fatty acids, dicarboxylic fatty acids, branched chain fatty acids, epoxy fatty acids or lactones.

Expression/overexpression of transporter proteins to increase secretion/production of fatty acids or fatty acid derived products such as fatty alcohols, fatty aldehydes, fatty esters, hydrocarbons, triacylglycerides, hydroxy fatty acids, dicarboxylic fatty acids, branched chain fatty acids, epoxy fatty acids or lactones, can be combined with any of the embodiments outlines above, or be completely independent.

In an embodiment, the fungal cell is capable of producing more than 100 mg of fatty acids per L of culture medium, and/or more than 10 mg of fatty acids per g dry cell weight (DCW).

In a particular embodiment, the fungal cell is capable of producing more than 250 mg, preferably more than 500 mg, and more preferably more than 750 mg, such as more than 1 g of fatty acids per L of culture medium.

In an alternative or additional particular embodiment, the fungal cell is capable of producing more than 15 mg, preferably more than 25 mg, and more preferably more than 30 mg fatty acid per g CDW.

The above described embodiments may be combined.

Other aspects of the invention provide methods for the production of fatty acids and/or fatty acid-derived products. Such methods comprises culturing a fungal cell according to any of the embodiments in a culture medium and in culture conditions suitable for production of the fatty acid and/or fatty acid-derived product by the fungal cell. The method also comprises collecting the fatty acid and/or fatty acid-derived product from the culture medium and/or the fungal cell.

The fatty acid-derived product is preferably selected from the group consisting of fatty alcohols, fatty aldehydes, fatty esters, hydrocarbons, triacylglycerides, lactones, phospholipids and a mixture thereof, preferably from the group consisting of fatty alcohols, fatty aldehydes, fatty esters, and a mixture thereof, and more preferably from the group consisting of hydroxy fatty acids, fatty alcohols, fatty aldehydes, and a mixture thereof.

In an embodiment, the culture medium is nitrogen-limited.

In an embodiment, the production process is composed of a growth phase, where the fungal cell is cultivated in the presence of high levels of the carbon source, e.g., glucose, and a production phase, where the fungal cell is cultivated in limiting conditions of the carbon source. This can be achieved, for example in a fed-batch process.

### EXAMPLES

### Example 1: Metabolic Engineering of the Acetyl-CoA Supply Results in High Production of Free Fatty Acids (FFA) in Fungal Cells

This example shows that fatty acid production in a fungal cell can be increased by improving the conversion of pyruvate to acetyl-CoA through novel modifications in the acetyl-CoA metabolism. In particular, mitochondrial citrate synthesis was enhanced. The resulting citrate could be used by the enzyme ATP:citrate lyase (ACL), which cleaves citrate to oxaloacetate and acetyl-CoA. Acetyl-CoA is in turn used for the production of fatty acids. Acetyl-CoA Carboxylase (ACC) catalyzes the first step in fatty acids formation from acetyl-CoA.

Genetic modifications in yeast were carried out via promoter replacement, deletion of genes and integration of expression cassettes. Standard molecular biology methods were used, including the use of integration cassettes, use of the selective markers Ura, His and Kanamycin and marker loop out as described in David and Siewers, 2015.

As background yeast strain the strain YJZ45 (CEN.PK 113-11C (MATa; MAL2-8c; SUC2; his3Δ1; ura3-52; hfd1Δ; pox1Δ; faa1Δ; faa4Δ; ura3Δ::HIS3+ MmACL+RtME+CTP1+′MDH3+tTesA+′tesA; URA3A::RtFAS1+RtFAS2+amdSym)) was used. Genetic modifications included promoter replacement in front of various genes including PYC1 (from −200 bp to 0 bp), ACC1 (from −481 bp to 0 bp), MP3, MP2, YHM2 replacing the native promoter with the constitutive active TEF1, PGK1 and TPI promoter, respectively. Heterologous expression of AnACL and RtCIT1 was facilitated via genomic integration of GAL1p-ACLa and GAL10p-ACLb, and HXT7p-RtCIT1 expression cassettes.

Yeast strains for preparation of competent cells were cultivated in YPD consisting of 10 g/L yeast extract (Merck Millipore, Billerica, MA, USA), 20 g/L peptone (Difco) and 20 g/L glucose (Merck Millipore). Constructed plasmids and integration cassettes were transformed into respective yeast strains via the Lithium acetate method as previously described (Gietz et al., 2007). Strains containing URA3-based plasmids or cassettes were selected on synthetic complete media without uracil (SC-URA), which consisted of 6.7 g/L yeast nitrogen base (YNB) without amino acids (Formedium, Hunstanton, UK), 0.77 g/L complete supplement mixture without uracil (CSM-URA, Formedium), 20 g/L glucose (Merck Millipore) and 20 g/L agar (Merck Millipore). The URA3 maker was removed and selected against on 5-FOA plates, which contained 6.7 g/L YNB, 0.77 g/L CSM-URA and 0.8 g/L 5-fluoroorotic acid. Shake flask batch fermentations for production of free fatty acids were carried out in minimal medium containing 2.5 g/L (NH4)₂SO₄, 14.4 g/L KH₂PO₄, 0.5 g/L MgSO₄·7H₂O, 30 g/L glucose, trace metal and vitamin solutions supplemented with 60 mg/L uracil if needed. Cultures were inoculated, from 24 h precultures, at an initial OD₆₀₀ of 0.1 with 15 ml minimal medium in 100 mL unbaffled flask and cultivated at 200 rpm, 30° C. for 72 h. Glucose feed beads (SMFB63319, Kuhner Shaker, Basel, Switzerland) with a release rate of 0.25 g/L/h were added to the medium to replace the 30 g/L glucose if needed, and the culture time is 80 h for totally release the glucose. For nitrogen restricted culture, 1.4 g/L (NH4)₂SO₄ were used.

FFA titers in whole-cell culture (only FFA was measured in this study) were quantified following previously published methods (Zhou et al., 2016). Specifically, 0.2 ml of cell culture (or an appropriate volume of cell culture diluted to 0.2 ml) were transferred to glass vials from 72 h or 80 h incubated cultures, then 10 ml 40% tetrabutylammonium hydroxide (base catalyst) was added immediately followed by addition of 200 ml dichloromethane containing 200 mM methyl iodide as methyl donor and 100 mg/L pentadecanoic acid as an internal standard. The mixtures were shaken for 30 min at 1,200 rpm by using a vortex mixer, and then centrifuged at 4,000×g to promote phase separation. A 150 ml dichloromethane layer was transferred into a GC vial with glass insert, and evaporated 3 h to dryness. The extracted methyl esters were resuspended in 150 ml hexane and then analyzed by gas chromatography (Focus GC, Thermo Fisher Scientific) equipped with a Zebron ZB-5MS GUARDIAN capillary column (30m×0.25 mm×0.25 mm, Phenomenex) and a DSQII mass spectrometer (Thermo Fisher Scientific). The GC program was as follows: initial temperature of 40° C., hold for 2 min; ramp to 130° C. at a rate of 30° C. per minute, then raised to 280° C. at a rate of 10° C. per min and hold for 3 min. The temperatures of inlet, mass transfer line and ion source were kept at 280, 300 and 230° C., respectively. The injection volume was 1 μl. The flow rate of the carrier gas (helium) was set to 1.0 ml/min, and data were acquired at full-scan mode (50-650 m/z). Final quantification was performed using the Xcalibur software.

The extracellular glucose, glycerol, ethanol and organic acid concentrations were determined by high-performance liquid chromatography analysis. In detail, a 1.5 ml broth sample was filtered through a 0.2 mm syringe filter and analyzed on an Aminex HPX-87G column (Bio-Rad) on an Ultimate 3000 HPLC (Dionex Softron GmbH). The column was eluted with 5 mM H₂SO₄ at a flow rate of 0.6 ml/min at 45° C. for 26 min.

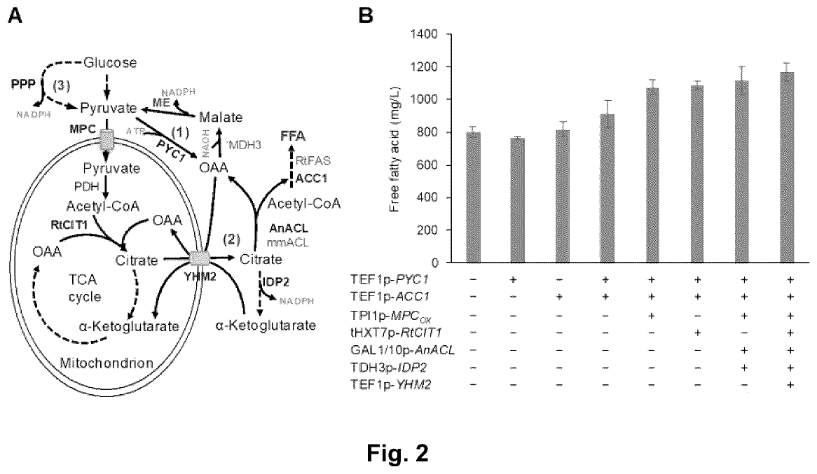

Overexpression of pyruvate carboxylase (PYC1) to ensure efficient formation of oxaloacetate required for citrate production did not result in a significant increase in FFA production (FIG. 2B). Neither did overexpression of acetyl-CoA carboxylase (ACC1) (FIG. 2B). However, combination of both modifications surprisingly resulted in a 14% increase in production of free fatty acids (FIG. 2B). When MPC1 and MPC3 (which together form the MPCₒₓ pyruvate transport complex) were overexpressed in a strain overexpressing PYC1 and ACC1 in order to improve transport of pyruvate to the mitochondria, a further 17% improvement in production was observed (FIG. 2B). When citrate synthase from Rhodosporidium toruloides (RtCIT1) was overexpressed in a strain overexpressing PYC1 and ACC1, a further 18% improvement in fatty acid titers was observed compared to the parental strain (FIG. 2B). Combining overexpression of PYC1, ACC1, MPC1, MPC3 and YHM2 with heterologous expression of AnACL and RtCIT1 lead to a 256% increase in FFA production compared to a strain only overexpressing PYC1 and ACC1, and a 46% improvement compared to the starting strain (FIG. 2B).

### Example 2: Further Fine-Tuning of Gene Expression Improves Free Fatty Acid (FFA) Production by Fungal Cells

Additional engineering was done through fine tuning of gene expression of the gene PGI1 (from −405 bp to 0 bp) involved in glycolysis and IDH2 (from −456 bp to 0 bp) in TCA cycle through promoter replacement with promoters displaying lower activity. All genetic modifications, cultivations, and analysis were performed as described in Example 1.

For PGI1, the promoter replacement was done through integration cassettes with the promoters of ISU1 (SEQ ID NO: 15), ATP14 (SEQ ID NO: 16), QCR10 (SEQ ID NO: 17), COX9 (SEQ ID NO: 18), NAT1 (SEQ ID NO: 19) and HXT1 (SEQ ID NO: 20), which were amplified from CEN.PK113-5D genomic DNA. Additionally integration cassettes were constructed for overexpression of Pentose Phosphate Pathway associated genes, including: P_{HX71}−TKL1, P_{PGK1}−TAL1, P_{TEF1}−ZWF1, P_{TDH3}−GND1.

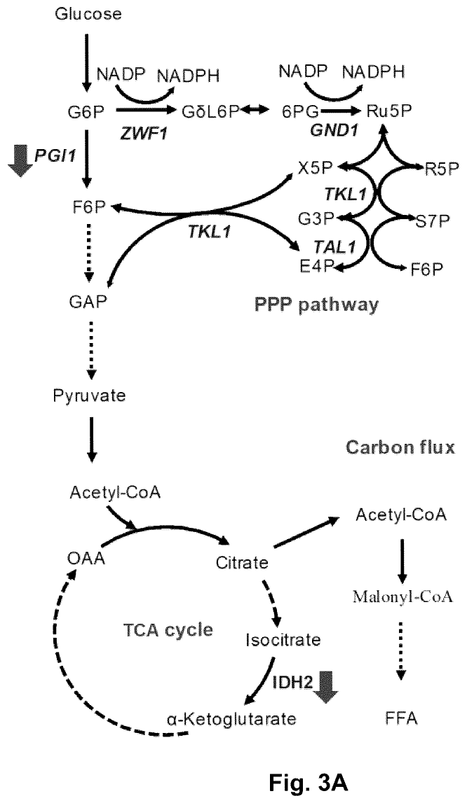

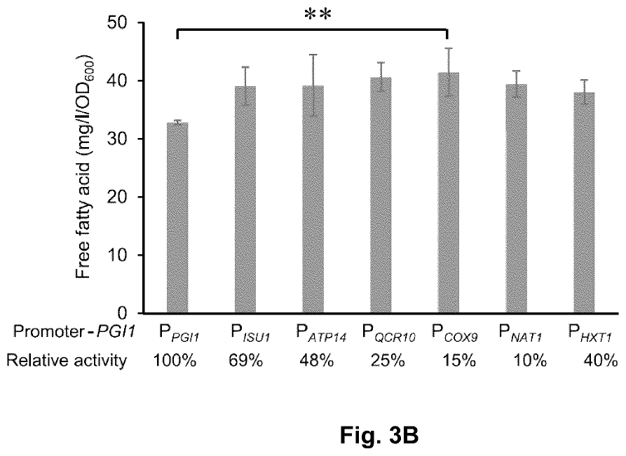

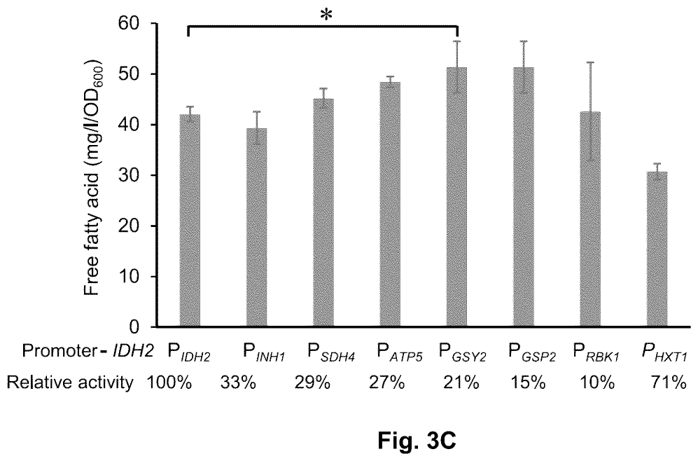

Replacing the native promoter of PGI1 with a weaker promoter increased FFA production by 20-27%, with the COX9 promoter displaying the best results (FIG. 3B).

For IDH2, the promoter replacement was done through integration cassettes with the promoters of INH1 (SEQ ID NO: 21), SDH4 (SEQ ID NO: 22), ATP5 (SEQ ID NO: 23), GSY2 (SEQ ID NO: 24), GSP2 (SEQ ID NO: 25), RBK1 (SEQ ID NO: 26) and HXT1 (SEQ ID NO: 20), which were amplified from CEN.PK113-5D genomic DNA. Replacing the native promoter of IDH2 with a weaker promoter increased FFA production by up to 22%, with the GSY2 and GSP2 promoters displaying the best results (FIG. 3C).

The resulting strain TY36 having the following genetic background: MATa; MAL2-8c; SUC2; his3Δ1; ura3-52; hfd1Δ; pox1Δ; faa1Δ; faa4Δ; ura3Δ::HIS3+MmACL+RtME+CTP1+′MDH3+tTesA+′tesA; URA3A::RtFAS1+RtFAS2+amdSym; acc1::TEF1p-ACC1; pyc1::TEF1p-PYC1; X1-4::MPC1+MPC3; gal80Δ; X1-2::AnACL; gal1Δgal7Δgal10Δ::RtCIT1+IDP2+YHM2; pgi1Δ::COX9p-PGI1+GND1+TKL1+TAL1+ZWF1; idh2Δ::GSY1p-IDH2, which combined all modifications was tested under fed-batch conditions (see Example 3).

### Example 3: Growth-Production De-Coupling

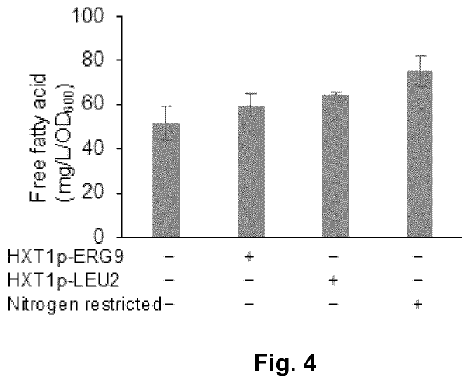

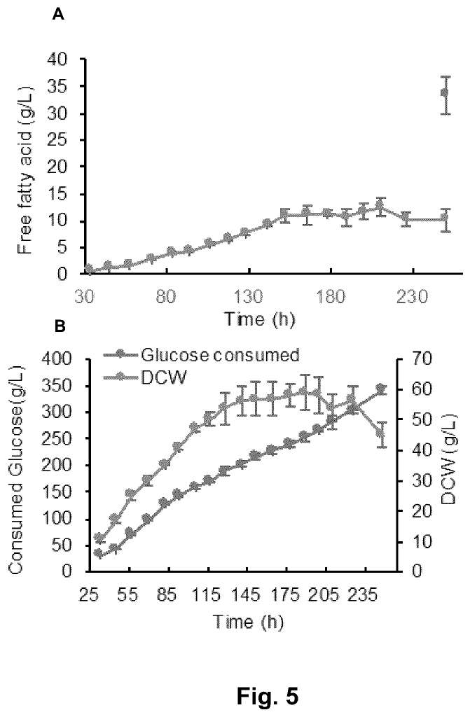

In oleaginous fungi, lipid overproduction is always initiated by growth stagnation that is triggered by limitation of nutrients such as nitrogen, which is due to the fact that biomass formation competes for carbon and energy. We therefore decoupled FFA production from cell growth through limiting cell growth by dynamically controlling the expression of essential genes under the HXT1 promoter, whereby we can tune cell growth by controlling the glucose concentration. The native promoter of LEU2 (from −195 bp to 0 bp) and ERG9 (from −138 bp to 0 bp) were replaced by the HXT1 promoter using standard techniques involving integration cassettes, marker selection and removal as previously described in Example 1. Genetic modifications, cultivation and analytics were carried out as described in Example 1. The promoter replacement lead to 15% increase in free fatty acid production in case of the ERG9 promoter and 25% for the LEU2 promoter (FIG. 4). Cultivation of this particular strain without any promoter replacement regarding LEU2 and ERG9 but under nitrogen limitation lead to 47% increase in free fatty acid production (FIG. 4). A fed-batch cultivation of strain TY36 under nitrogen limiting conditions was carried out leading to very high titers of free fatty acids of 35 g/L (FIG. 5). The batch and fed-batch fermentations for free fatty acid production were performed in 1.0 L bioreactors, with an initial working volume of 0.25 L, in a DasGip Parallel Bioreactors System (DasGip). The initial batch fermentation was carried out in minimal medium containing 5 g/L (NH4)₂SO₄, 3 g/L KH₂PO₄, 0.5 g/L MgSO₄·7H₂O, 60 mg/L URA, 20 g/L glucose, trace metal and vitamin solutions. The temperature, agitation, aeration and pH were monitored and controlled using a DasGip Control 4.0 System. The temperature was kept at 30° C., initial agitation set to 800 rpm and increased to maximally 1,200 rpm depending on the dissolved oxygen level. Aeration was initially provided at 36 sl/h and increased to maximally 48 sl/h depending on the dissolved oxygen level. The dissolved oxygen level was maintained above 30%, the pH was kept at 5.6 by automatic addition of 4 M KOH and 2 M HCl. The aeration was controlled and provided by a DasGip MX4/4 module. The composition of the off-gas was monitored using a DasGip Off gas Analyzer GA4. Addition of the acid, base, and glucose feed was carried out with DasGip MP8 multi-pump modules (pump head tubing: 0.5 mm ID, 1.0 mm wall thickness).

The pumps, pH and DO probes were calibrated before the experiment. During the fed-batch cultivation, the cells were initially fed with a 200 g/L glucose solution with a feed rate that was exponentially increased (p=0.05/h) to maintain a constant biomass-specific glucose consumption rate. The used minimal medium contained 15 g/L (NH₄)₂SO₄, 9 g/L KH₂PO₄, 1.5 g/L MgSO₄·7H₂O, 180 mg/L uracil, 3× trace metal and 3× vitamin solution. When the volume of the fermentation broth reached 0.4-0.45 L, the feed solution was switched to the following composition: 25 g/L (NH₄)₂SO₄, 15 g/L KH₂PO₄, 2.5 g/L MgSO₄·7H₂O, 300 mg/L uracil, 600 g/L glucose, 5× trace metal and 5× vitamin solution. The initial feed rate was calculated using the biomass yield and concentration that were obtained during prior duplicate batch cultivations with these strains. The feeding was started once the dissolved oxygen level was higher than 30%. Dry cell weight measurements were performed by filtrating 3-5 ml of broth through a weighed 0.45 mm filter membrane (Sartorius Biolab, Gottingen, Germany) and measuring the weight increase after drying for 48 h in a 65° C. oven. The filter was washed once before and three times after filtrating the broth with 5 ml deionized water. During fermentation, floating dead cells and fatty acid residues were found to stick to the inner wall or the bottom of the fermenter. After fermentation, all particles were resuspended in the fermentation culture to accurately measure the total FFA production. Measurements were performed three times.

### Example 4: Abolishment of Ethanol Production

Ethanol is often a side-product of fermentation and might be undesired if production of fatty acids is the main goal. Pyruvate decarboxylases (PDC1, PDC5, and PDC6) catalyze the decarboxylation of pyruvate to acetaldehyde, which plays a key role in alcoholic fermentation in S. cerevisiae. Deletion of these genes leads to abolishment of ethanol production. However, a PDC-negative strain with a triple deletion of all PDC genes is unable to grow on glucose as the sole carbon source.

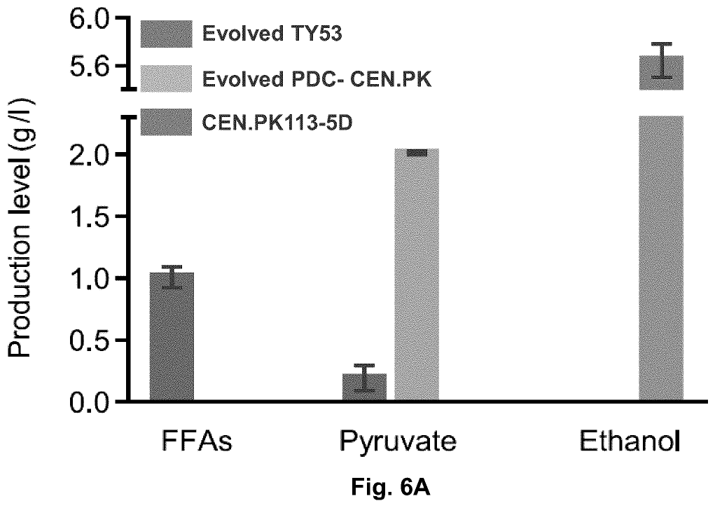

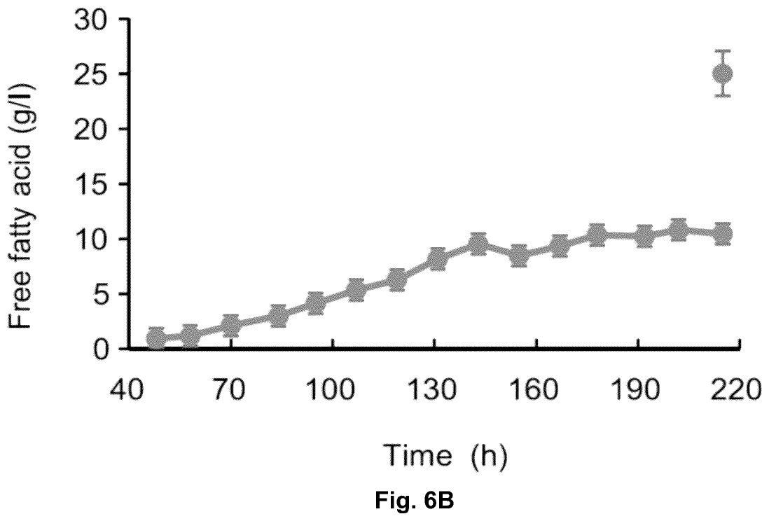

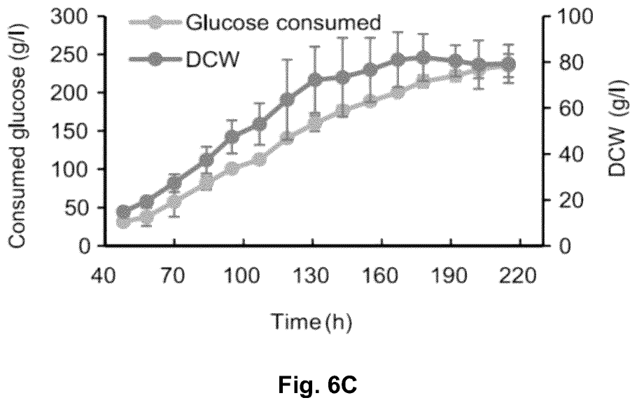

PDC1, PDC5 and PDC6 were deleted from the fatty acid-producing strain TY36 using the methodology outlined in Example 1, resulting in strain TY53. However, the resulting strain was unable to grow on glucose. In order to facilitate growth on glucose, the strain was evolved using Adaptive Laboratory Evolution. The adaptive evolution of TY53 (TY36 pdc1Δ, pdc5Δ, pdc6Δ) toward growth on glucose as the sole carbon source were performed in three independent culture lines in 100 mL shake flasks with 15 mL medium at 30C, which involved two phases. In the first phase, strains were cultivated in minimal medium containing 0.5% glucose and 2% ethanol and then serially transferred every 48 or 72 h using minimal medium with a gradually decreased ethanol concentration and increased glucose concentration for 45 days. Subsequently, the strains were transferred into minimal medium containing 2% glucose as the sole carbon source and evolved for increased growth by serial transfer every 48 or 72 h for 50 days. Several strains were isolated from the evolved populations and tested. The evolved TY53 strains could grow on glucose. The performance of the evolved strains was compared to a wild-type strain (CEN.PK113-5D) and an evolved PDC-negative wild-type strain (evolved PDC-CEN.PK) (FIG. 6A). When cultured under shake-flask conditions (as described in Example 1), the evolved TY53 strains could produce fatty acids, produced less pyruvate than evolved PDC-CEN.PK and did not produce ethanol (FIG. 6A). When grown under fed-batch conditions (performed as described in Example 3) the strains produced up to 25 g/L of free fatty acids (FIG. 6B) and accumulated biomass (FIG. 6C)

To evaluate the underlying mechanisms, total genomic DNA of selected strains was extracted by using the Blood & Cell Culture DNA Kit (QIAGEN). Then DNA was prepared using the Illumina TruSeq Nano DNA HT 96 protocol, according to the manufacturer's instructions. The samples were sequenced using an Illumina NextSeq High kit, paired-end 300 cycles (2×150 bp). Each sample was represented by 2.2-6.4 million sequence reads. Breseq (Deatherage and Barrick, 2014) 0.30.2 with bowtie (Langmead and Salzberg, 2012) 2.2.8 was used to map the reads of each sample to the genome of S. cerevisiae CEN.PK 113-7D (Jenjaroenpun et al., 2018). The option junction-alignment-pair-limit set to 0 (no limit) to ensure all possible new junctions were evaluated. The sequencing data for the initial strain (TY36) was also processed with breseq and used as a reference for removing false-positives from the sample analysis.

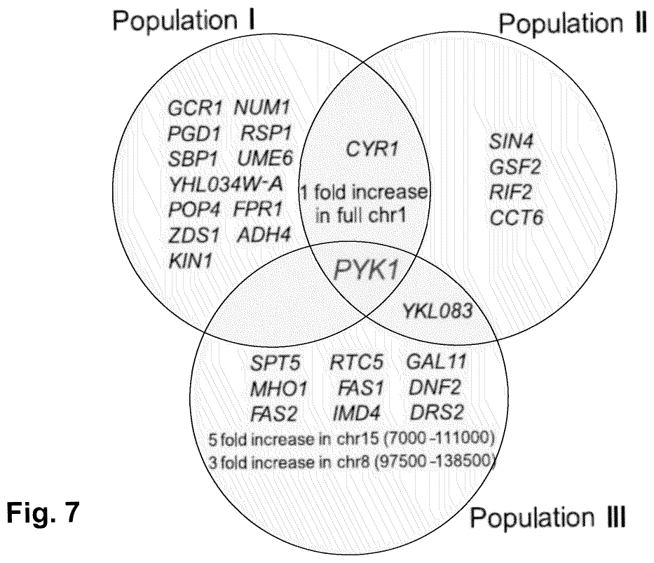

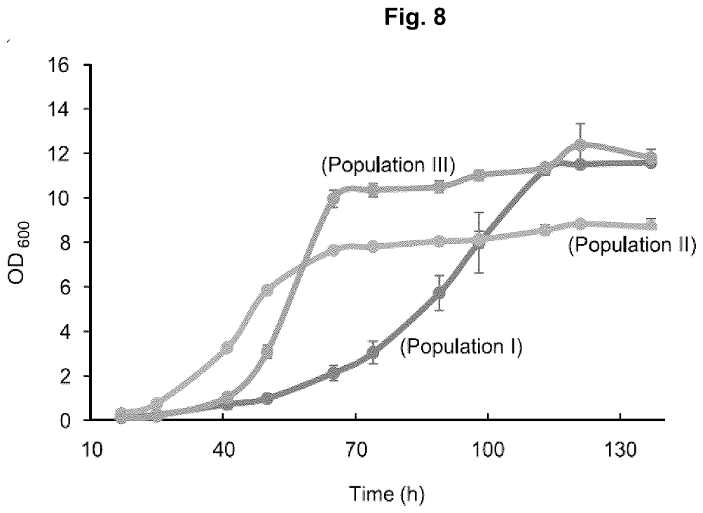

The mutations found in the three clones are shown in FIG. 7. The growth of each clone (under shake-flask conditions, as described in Example 1) is shown in FIG. 8.

It was found that mutations in pyruvate kinase (PYK1), also known as CDC19 in S. cerevisiae, occurred in all three evolved clones: two nonsense mutations (R68* and K196*) and a missense mutation (R911). PYK1 (CDC19) is the major pyruvate kinase which converts phosphoenolpyruvate (PEP) and ADP to pyruvate and ATP. PYK1 is tightly regulated and activated by fructose-1,6-bisphosphate (FBP) and considered as a key control point of glycolytic flux. The mutations in PYK1 across the evolved mutants suggested that decease in PYK1 activity is important for growth on glucose.

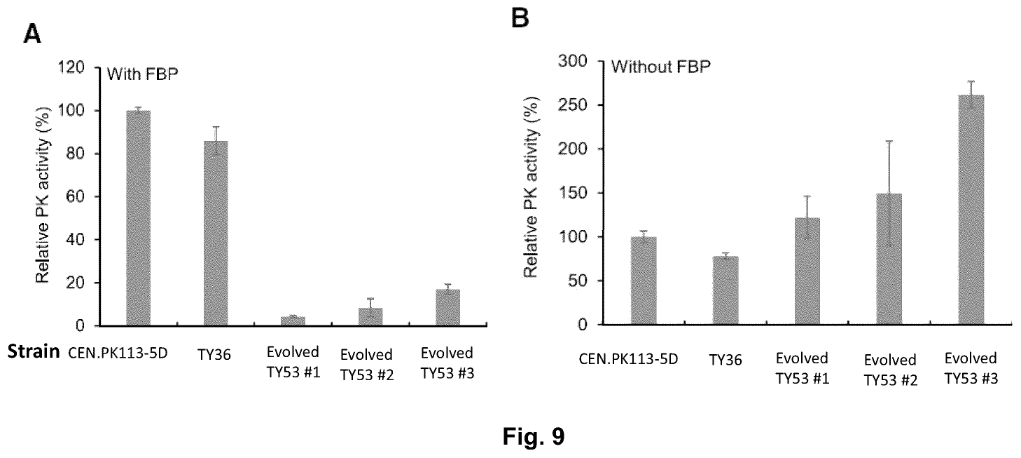

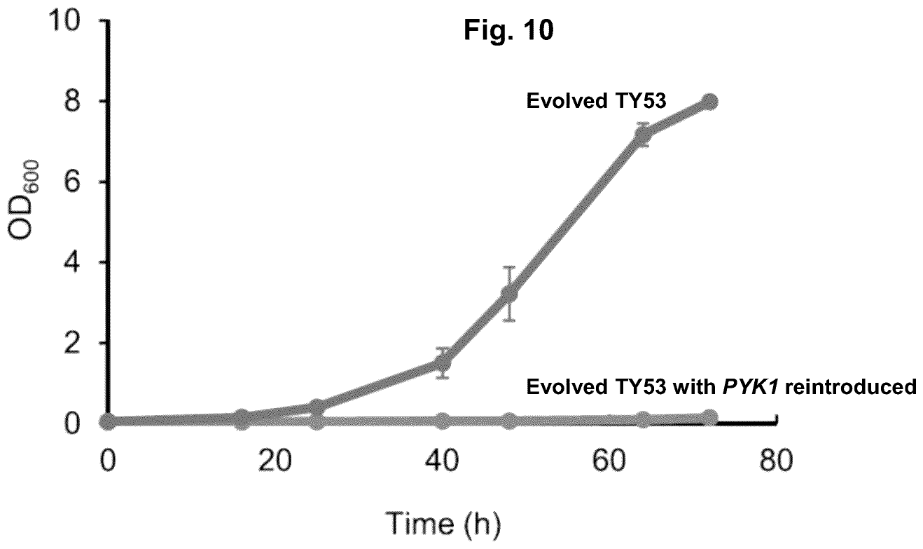

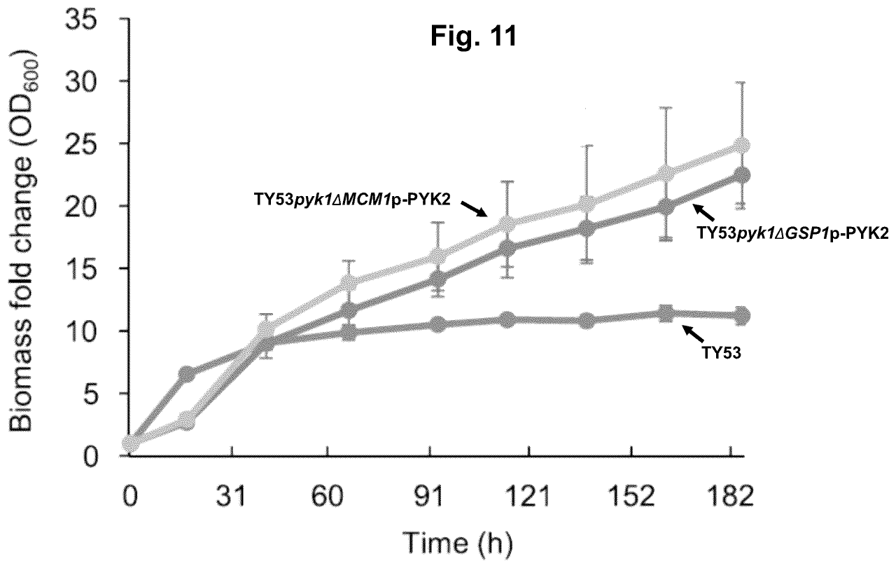

In addition, it was found that while the evolved mutants had a much lower PYK activity in general, they had a higher PYK2 activity (FIG. 9) compared to the unevolved strain and PYK2 was found to be increased in copy number in the evolved strains. In order to confirm the role of PYK in growth on glucose, wild-type PYK1 was reintroduced into the evolved strain, which resulted in abolishment of growth in glucose medium (FIG. 10). In addition, we found that deletion of PYK1 and overexpression of PYK2 in the PDC-negative strain TY53 enabled growth on glucose (FIG. 11).

Overall, these results demonstrate that it is possible to abolish production of the by-product ethanol and maintain growth on glucose by deleting the PDC genes, down-regulating PYK1 and overexpressing PYK2.

### Example 5: Overexpression of Endogenous Genes

This example demonstrates that increased production of fatty acids can be achieved through overexpression of selected endogenous genes.

The endogenous genes MPP6, ACP1, EPT1, FAA1, GEP4, GGA2, IDP3, INP54, LPP1, MCR1, ORM1, RTC3, SPO7, TGL1 and YFT2 were amplified from the genomic DNA of S. cerevisiae strain IMX581 (derived from the strain CEN.PK113-5D Mans et al., 2015). The genes were integrated into the integration site X_3 (Jessop-Fabre et al., 2016) in the background strain IMX581. Promoter PTEF1 and terminator TCYC1 were used for controlling gene expression of the selected genes. Amplified genetic parts, including homologous regions and promoter-gene-terminator, were assembled into a cassette through a two-step fusion PCR procedure adapted from (Zhou et al., 2012) and transformed into strain IMX581. This resulted in 15 S. cerevisiae strains, each overexpressing one of the following genes: MPP6, ACP1, EPT1, FAA1, GEP4, GGA2, IDP3, INP54, LPP1, MCR1, ORM1, RTC3, SPO7, TGL1 and YFT2.

The above strains, as well as a control strain (IMX581) not overexpressing any of the endogenous genes mentioned above, were inoculated from 48 h pre-cultures at an OD₆₀₀ of 0.1 in 25 mL minimal medium (described in Example 1) supplemented with 60 mg/L uracil in 100 mL shake flasks. Strains were cultivated at 30° C. at 200 rpm. Samples for fatty acid analysis were taken after 48 hours of cultivation and processed as described in Example 1.

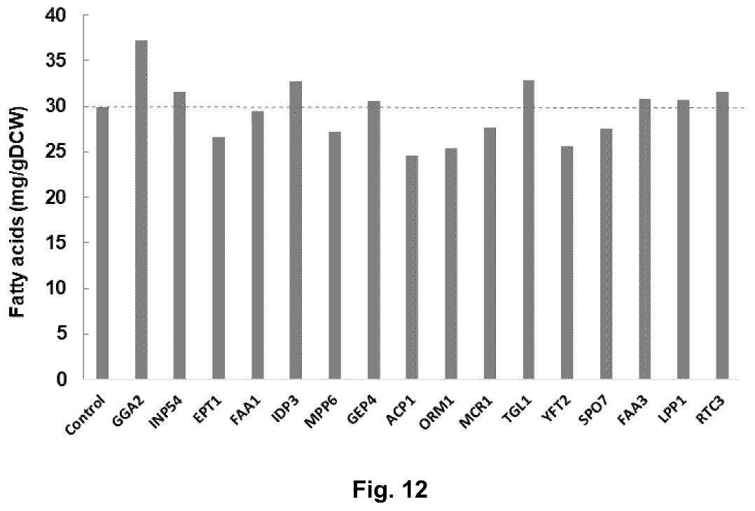

FIG. 12 shows the results for total fatty acid production as a consequence of overexpression of the genes described above. Individual overexpression of GGA2, INP54, IDP3, GEP4, TGL1, FAA3, LPP1 and RTC3 showed a beneficial effect on fatty acid production. Strains overexpressing these genes displayed improved fatty acid production of 24.4%, 5.5%, 9.3%, 2.1%, 9.8%, 2.8%, 2.5% and 5.6% compared to the control strain, respectively (FIG. 12). These results show that overexpression of these genes is a good strategy to improve production of fatty acids in yeast, with overexpression of GGA2 being the most promising strategy.

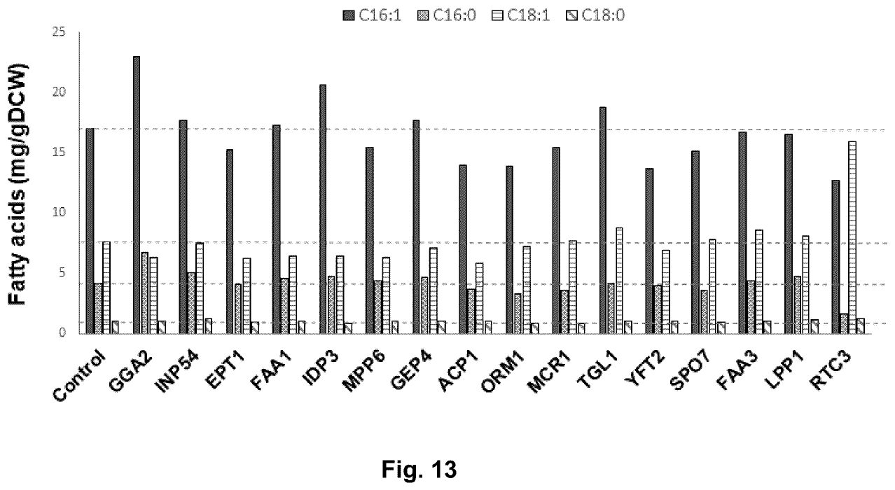

While for some applications production of a mixture of different fatty acids is desirable, in some cases production of specific fatty acids is preferred. Therefore, in addition to measuring total fatty acids, the effects of the overexpression of the aforementioned endogenous genes on production of specific fatty acids, including palmitic acid (C16:0), palmitoleic acid (C16:1), stearic acid (C18:0) and oleic acid (C16:1) were quantified. These results are depicted in FIG. 13. It was found that individual overexpression of GGA2, INP54, FAA1, IDP3, GEP4, FAA3, and LPP1 increased production of palmitic acid (C16:0) by 60.6%, 21.4%, 8%, 13.5%, 10.4%, 3% and 13.3% compared to control, respectively (FIG. 13). It was also found that individual overexpression of GGA2, INP54, IDP3, GEP4 and TGL1 increased production of palmitoleic acid (C16:1) by 35.4%, 4.2%, 21.2%, 3.9% and 10.5% compared to control, respectively (FIG. 13). In addition, individual overexpression of TGL1, SPO7, FAA3, and RTC3 increased production of oleic acid (C18:1) by 14.5%, 2.2%, 12% and 107.8% compared to the control, respectively (FIG. 13). Finally, individual overexpression of GGA2, INP54, FAA1, MPP6, GEP4, TGL1, FAA3, LPP1 and RTC3 increased production of stearic acid (C18:0) by 6.6%, 19.5%, 2.8%, 4.8%, 3%, 2.7%, 4.7%, 14.1% and 24.6% compared to control, respectively. These results indicated that in addition to having beneficial effects on the production of fatty acids in general, several of these genes had beneficial effects on production of specific fatty acids. In particular, production of palmitic acid particularly benefited from overexpression of GGA2 or INP54, production of palmitoleic acid particularly benefited from overexpression of GGA2 or IDP3, production of oleic acid particularly benefited from overexpression of RTC3, and production of stearic acid particularly benefited from overexpression of INP54 or RTC3.

### REFERENCES

David and Siewers, Advances in yeast genome engineering, FEMS yeast research, 2015, 15(1): 1-14
Deatherage and Barrick, Identification of mutations in laboratory-evolved microbes from next-generation sequencing data using breseq, Methods Mol. Biol, 2014, 1151: 165-188
Gietz and Schiestl, High-efficiency yeast transformation using the LiAc/SS carrier DNA/PEG method, Nature Protocols, 2007, 2(1): 31-34
Jenjaroenpun et al., Complete genomic and transcriptional landscape analysis using third-generation sequencing: a case study of Saccharomyces cerevisiae CEN.PK113-7D, Nucleic Acids Res, 2018, 46: e38
Jessop-Fabre, et al., EasyClone-MarkerFree: A vector toolkit for marker-less integration of genes into Saccharomyces cerevisiae via CRISPR-Cas9, Biotechnol J, 2016, 11(8): 1110-1117
Langmead and Salzberg, Fast gapped-read alignment with Bowtie 2, Nat. Methods, 2012, 9: 357-359
Mans et al., CRISPR/Cas9: a molecular Swiss army knife for simultaneous introduction of multiple genetic modifications in Saccharomyces cerevisiae, FEMS Yeast Res, 2015, 15(2): pii: fov004
Pfleger et al., Metabolic engineering strategies for microbial synthesis of oleochemicals, Metabolic Engineering, 2015, 29: 1-11
Zhang et al., Adaptive mutations in sugar metabolism restore growth on glucose in a pyruvate decarboxylase negative yeast strain, Microbial cell factories, 2015, 14: 116
Zhou et al., Modular pathway engineering of diterpenoid synthases and the mevalonic acid pathway for miltiradiene production, J Am Chem Soc, 2012, 134(6): 3234-3241
Zhou et al., Production of fatty acid-derived oleochemicals and biofuels by synthetic yeast cell factories, Nature Communications, 2016, 7: 11709

1. A yeast cell for the production of fatty acids and/or fatty acid-derived products, wherein the yeast cell is genetically modified for overexpression of one or more endogenous yeast genes selected from the group consisting of ADP-ribosylation factor-binding protein GGA2 (SEQ ID NO: 13), a variant having an amino acid sequence at least 75% identical to SEQ ID NO: 13, phosphatidylinositol 4,5-bisphosphate 5-phosphatase INP54 (SEQ ID NO: 33), a variant having an amino acid sequence at least 75% identical to SEQ ID NO: 33, NADP-dependent isocitrate dehydrogenase IDP3 (SEQ ID NO: 32), a variant having an amino acid sequence at least 75% identical to SEQ ID NO: 32, mitochondrial phosphatidylglycerophosphatase GEP4 (SEQ ID NO: 31), a variant having an amino acid sequence at least 75% identical to SEQ ID NO: 31, triglyceride lipase TGL1 (SEQ ID NO: 39), a variant having an amino acid sequence at least 75% identical to SEQ ID NO: 39, long chain fatty acyl-CoA synthetase FAA3 (SEQ ID NO: 30), a variant having an amino acid sequence at least 75% identical to SEQ ID NO: 30, lipid phosphate phosphatase LPP1 (SEQ ID NO: 34), and a variant having an amino acid sequence at least 75% identical to SEQ ID NO: 34, wherein the yeast cell produces higher amount of fatty acids and/or fatty acid-derived products compared to a yeast cell not overexpressing the one or more endogenous yeast genes.

2. The yeast cell according to claim 1, wherein the yeast cell is genetically modified for overexpression of one or more endogenous yeast genes selected from the group consisting of GGA2 (SEQ ID NO: 13), the variant having an amino acid sequence at least 75% identical to SEQ ID NO: 13, IDP3 (SEQ ID NO: 32), the variant having an amino acid sequence at least 75% identical to SEQ ID NO: 32, TGL1 (SEQ ID NO: 39), and a variant having an amino acid sequence at least 75% identical to SEQ ID NO: 39.

3. The yeast cell according to claim 2, wherein the yeast cell is genetically modified for overexpression of GGA2 (SEQ ID NO: 13), or the variant having an amino acid sequence at least 75% identical to SEQ ID NO: 13.

4. The yeast cell according to claim 3, wherein the yeast cell is genetically modified for overexpression of GGA2 (SEQ ID NO: 13), the variant having an amino acid sequence at least 75% identical to SEQ ID NO: 13, IDP3 (SEQ ID NO: 32), or the variant having an amino acid sequence at least 75% identical to SEQ ID NO: 32.

5. The yeast cell according to claim 3, wherein the yeast cell is genetically modified for overexpression of GGA2 (SEQ ID NO: 13), the variant having an amino acid sequence at least 75% identical to SEQ ID NO: 13, TGL1 (SEQ ID NO: 39), or a variant having an amino acid sequence at least 75% identical to SEQ ID NO: 39.

6. The yeast cell from claim 1, wherein the yeast cell is selected from the group consisting of Saccharomyces, Kluyveromyces, Zygosaccharomyces, Candida, Hansenula, Torulopsis, Kloeckera, Pichia, Schizosaccharomyces, Trigonopsis, Brettanomyces, Debaromyces, Nadsonia, Lipomyces, Cryptococcus, Aureobasidium, Trichosporon, Lipomyces, Rhodotorula, Yarrowia, Rhodosporidium, Phaffia, Schwanniomyces, Aspergillus, and Ashbya.

7. The yeast cell according to claim 6, wherein the yeast cell is selected from the group consisting of Saccharomyces cerevisiae, Pichia pastoris, Ashbya gossypii, Saccharomyces boulardii, Zygosaccharomyces bailii, Kluyveromyces lactis, Rhodosporidium toruloides and Yarrowia lipolytica.

8. The yeast cell according to claim 1, wherein the yeast cell is genetically modified for overexpression of one or more endogenous yeast genes selected from the group consisting of GGA2 (SEQ ID NO: 13), a variant having an amino acid sequence at least 80% identical to SEQ ID NO: 13, INP54 (SEQ ID NO: 33), a variant having an amino acid sequence at least 80% identical to SEQ ID NO: 33, IDP3 (SEQ ID NO: 32), a variant having an amino acid sequence at least 80% identical to SEQ ID NO: 32, GEP4 (SEQ ID NO: 31), a variant having an amino acid sequence at least 80% identical to SEQ ID NO: 31, TGL1 (SEQ ID NO: 39), a variant having an amino acid sequence at least 80% identical to SEQ ID NO: 39, FAA3 (SEQ ID NO: 30), a variant having an amino acid sequence at least 80% identical to SEQ ID NO: 30, LPP1 (SEQ ID NO: 34), and a variant having an amino acid sequence at least 80% identical to SEQ ID NO: 34.

9. The yeast cell according to claim 1, wherein the yeast cell is genetically modified for overexpression of one or more endogenous yeast genes selected from the group consisting of GGA2 (SEQ ID NO: 13), a variant having an amino acid sequence at least 85% identical to SEQ ID NO: 13, INP54 (SEQ ID NO: 33), a variant having an amino acid sequence at least 85% identical to SEQ ID NO: 33, IDP3 (SEQ ID NO: 32), a variant having an amino acid sequence at least 85% identical to SEQ ID NO: 32, GEP4 (SEQ ID NO: 31), a variant having an amino acid sequence at least 85% identical to SEQ ID NO: 31, TGL1 (SEQ ID NO: 39), a variant having an amino acid sequence at least 85% identical to SEQ ID NO: 39, FAA3 (SEQ ID NO: 30), a variant having an amino acid sequence at least 85% identical to SEQ ID NO: 30, LPP1 (SEQ ID NO: 34), and a variant having an amino acid sequence at least 85% identical to SEQ ID NO: 34.

10. The yeast cell according to claim 1, wherein the yeast cell is genetically modified for overexpression of one or more endogenous yeast genes selected from the group consisting of GGA2 (SEQ ID NO: 13), a variant having an amino acid sequence at least 90% identical to SEQ ID NO: 13, INP54 (SEQ ID NO: 33), a variant having an amino acid sequence at least 90% identical to SEQ ID NO: 33, IDP3 (SEQ ID NO: 32), a variant having an amino acid sequence at least 90% identical to SEQ ID NO: 32, GEP4 (SEQ ID NO: 31), a variant having an amino acid sequence at least 90% identical to SEQ ID NO: 31, TGL1 (SEQ ID NO: 39), a variant having an amino acid sequence at least 90% identical to SEQ ID NO: 39, FAA3 (SEQ ID NO: 30), a variant having an amino acid sequence at least 90% identical to SEQ ID NO: 30, LPP1 (SEQ ID NO: 34), and a variant having an amino acid sequence at least 90% identical to SEQ ID NO: 34.

11. The yeast cell according to claim 1, wherein the yeast cell is genetically modified for overexpression of one or more endogenous yeast genes selected from the group consisting of GGA2 (SEQ ID NO: 13), a variant having an amino acid sequence at least 95% identical to SEQ ID NO: 13, INP54 (SEQ ID NO: 33), a variant having an amino acid sequence at least 95% identical to SEQ ID NO: 33, IDP3 (SEQ ID NO: 32), a variant having an amino acid sequence at least 95% identical to SEQ ID NO: 32, GEP4 (SEQ ID NO: 31), a variant having an amino acid sequence at least 95% identical to SEQ ID NO: 31, TGL1 (SEQ ID NO: 39), a variant having an amino acid sequence at least 95% identical to SEQ ID NO: 39, FAA3 (SEQ ID NO: 30), a variant having an amino acid sequence at least 95% identical to SEQ ID NO: 30, LPP1 (SEQ ID NO: 34), and a variant having an amino acid sequence at least 95% identical to SEQ ID NO: 34.

12. The yeast cell according to claim 1, wherein the yeast cell is genetically modified for overexpression of one or more endogenous yeast genes selected from the group consisting of GGA2 (SEQ ID NO: 13), a variant having an amino acid sequence at least 98% identical to SEQ ID NO: 13, INP54 (SEQ ID NO: 33), a variant having an amino acid sequence at least 98% identical to SEQ ID NO: 33, IDP3 (SEQ ID NO: 32), a variant having an amino acid sequence at least 98% identical to SEQ ID NO: 32, GEP4 (SEQ ID NO: 31), a variant having an amino acid sequence at least 98% identical to SEQ ID NO: 31, TGL1 (SEQ ID NO: 39), a variant having an amino acid sequence at least 98% identical to SEQ ID NO: 39, FAA3 (SEQ ID NO: 30), a variant having an amino acid sequence at least 98% identical to SEQ ID NO: 30, LPP1 (SEQ ID NO: 34), and a variant having an amino acid sequence at least 98% identical to SEQ ID NO: 34.

13. The yeast cell according to claim 12, wherein the yeast cell is genetically modified for overexpression of one or more endogenous yeast genes selected from the group consisting of GGA2 (SEQ ID NO: 13), INP54 (SEQ ID NO: 33), IDP3 (SEQ ID NO: 32), GEP4 (SEQ ID NO: 31), TGL1 (SEQ ID NO: 39), FAA3 (SEQ ID NO: 30), and LPP1 (SEQ ID NO: 34).

14. A method for producing a fatty acid comprising:
culturing a yeast cell according to claim 1 in a culture medium and in culture conditions suitable for production of the fatty acid by the yeast cell, and
collecting the fatty acid from the culture medium and/or the yeast cell.

15. A method for producing a fatty acid derived-product comprising:
culturing a yeast cell according claim 1 in a culture medium and in culture conditions suitable for production of the fatty acid-derived product by the yeast cell; and
collecting the fatty acid-derived product from the culture medium and/or the yeast cell, wherein said the acid-derived product is selected from the group consisting of a hydrocarbon, a triacylglyceride, a phospholipid, a lactone, a fatty alcohol, a fatty aldehyde, a fatty acid ester, and a mixture thereof.


SEQUENCE LISTING (40 sequences)

SEQ ID NO: 1 - protein, 2233 aa, Saccharomyces cerevisiae
MSEESLFESSPQKMEYEITNYSERHTELPGHFIGLNTVDKLEESPLRDFVKSHGGHTVISKILIANNGIAAVKEIRSVRKWAYETFGDDRTVQFVAMATPEDLEANAEYIRMADQYIEVPGGTNNNNYANVDLIVDIAERADVDAVWAGWGHASENPLLPEKLSQSKRKVIFIGPPGNAMRSLGDKISSTIVAQSAKVPCIPWSGTGVDTVHVDEKTGLVSVDDDIYQKGCCTSPEDGLQKAKRIGFPVMIKASEGGGGKGIRQVEREEDFIALYHQAANEIPGSPIFIMKLAGRARHLEVQLLADQYGTNISLFGRDCSVQRRHQKIIEEAPVTIAKAETFHEMEKAAVRLGKLVGYVSAGTVEYLYSHDDGKFYFLELNPRLQVEHPTTEMVSGVNLPAAQLQIAMGIPMHRISDIRTLYGMNPHSASEIDFEFKTQDATKKQRRPIPKGHCTACRITSEDPNDGFKPSGGTLHELNFRSSSNVWGYFSVGNNGNIHSFSDSQFGHIFAFGENRQASRKHMVVALKELSIRGDFRTTVEYLIKLLETEDFEDNTITTGWLDDLITHKMTAEKPDPTLAVICGAATKAFLASEEARHKYIESLQKGQVLSKDLLQTMFPVDFIHEGKRYKFTVAKSGNDRYTLFINGSKCDIILRQLSDGGLLIAIGGKSHTIYWKEEVAATRLSVDSMTTLLEVENDPTQLRTPSPGKLVKFLVENGEHIIKGQPYAEIEVMKMQMPLVSQENGIVQLLKQPGSTIVAGDIMAIMTLDDPSKVKHALPFEGMLPDFGSPVIEGTKPAYKFKSLVSTLENILKGYDNQVIMNASLQQLIEVLRNPKLPYSEWKLHISALHSRLPAKLDEQMEELVARSLRRGAVFPARQLSKLIDMAVKNPEYNPDKLLGAVVEPLADIAHKYSNGLEAHEHSIFVHFLEEYYEVEKLFNGPNVREENIILKLRDENPKDLDKVALTVLSHSKVSAKNNLILAILKHYQPLCKLSSKVSAIFSTPLQHIVELESKATAKVALQAREILIQGALPSVKERTEQIEHILKSSVVKVAYGSSNPKRSEPDLNILKDLIDSNYVVFDVLLQFLTHQDPVVTAAAAQVYIRRAYRAYTIGDIRVHEGVTVPIVEWKFQLPSAAFSTFPTVKSKMGMNRAVSVSDLSYVANSQSSPLREGILMAVDHLDDVDEILSQSLEVIPRHQSSSNGPAPDRSGSSASLSNVANVCVASTEGFESEEEILVRLREILDLNKQELINASIRRITFMFGFKDGSYPKYYTFNGPNYNENETIRHIEPALAFQLELGRLSNFNIKPIFTDNRNIHVYEAVSKTSPLDKRFFTRGIIRTGHIRDDISIQEYLTSEANRLMSDILDNLEVTDTSNSDLNHIFINFIAVFDISPEDVEAAFGGFLERFGKRLLRLRVSSAEIRIIIKDPQTGAPVPLRALINNVSGYVIKTEMYTEVKNAKGEWVFKSLGKPGSMHLRPIATPYPVKEWLQPKRYKAHLMGTTYVYDFPELFRQASSSQWKNFSADVKLTDDFFISNELIEDENGELTEVEREPGANAIGMVAFKITVKTPEYPRGRQFVVVANDITFKIGSFGPQEDEFFNKVTEYARKRGIPRIYLAANSGARIGMAEEIVPLFQVAWNDAANPDKGFQYLYLTSEGMETLKKFDKENSVLTERTVINGEERFVIKTIIGSEDGLGVECLRGSGLIAGATSRAYHDIFTITLVTCRSVGIGAYLVRLGQRAIQVEGQPIILTGAPAINKMLGREVYTSNLQLGGTQIMYNNGVSHLTAVDDLAGVEKIVEWMSYVPAKRNMPVPILETKDTWDRPVDFTPTNDETYDVRWMIEGRETESGFEYGLFDKGSFFETLSGWAKGVVVGRARLGGIPLGVIGVETRTVENLIPADPANPNSAETLIQEPGQVWHPNSAFKTAQAINDFNNGEQLPMMILANWRGFSGGQRDMFNEVLKYGSFIVDALVDYKQPIIIYIPPTGELRGGSWVVVDPTINADQMEMYADVNARAGVLEPQGMVGIKFRREKLLDTMNRLDDKYRELRSQLSNKSLAPEVHQQISKQLADRERELLPIYGQISLQFADLHDRSSRMVAKGVISKELEWTEARRFFFWRLRRRLNEEYLIKRLSHQVGEASRLEKIARIRSWYPASVDHEDDRQVATWIEENYKTLDDKLKGLKLESFAQDLAKKIRSDHDNAIDGLSEVIKMLSTDDKEKLLKTLK

SEQ ID NO: 2 - protein, 1178 aa, Saccharomyces cerevisiae
MSQRKFAGLRDNFNLLGEKNKILVANRGEIPIRIFRTAHELSMQTVAIYSHEDRLSTHKQKADEAYVIGEVGQYTPVGAYLAIDEIISIAQKHQVDFIHPGYGFLSENSEFADKVVKAGITWIGPPAEVIDSVGDKVSARNLAAKANVPTVPGTPGPIETVEEALDFVNEYGYPVIIKAAFGGGGRGMRVVREGDDVADAFQRATSEARTAFGNGTCFVERFLDKPKHIEVQLLADNHGNVVHLFERDCSVQRRHQKVVEVAPAKTLPREVRDAILTDAVKLAKECGYRNAGTAEFLVDNQNRHYFIEINPRIQVEHTITEEITGIDIVAAQIQIAAGASLPQLGLFQDKITTRGFAIQCRITTEDPAKNFQPDTGRIEVYRSAGGNGVRLDGGNAYAGTIISPHYDSMLVKCSCSGSTYEIVRRKMIRALIEFRIRGVKTNIPFLLTLLTNPVFIEGTYWTTFIDDTPQLFQMVSSQNRAQKLLHYLADVAVNGSSIKGQIGLPKLKSNPSVPHLHDAQGNVINVTKSAPPSGWRQVLLEKGPAEFARQVRQFNGTLLMDTTWRDAHQSLLATRVRTHDLATIAPTTAHALAGRFALECWGGATFDVAMRFLHEDPWERLRKLRSLVPNIPFQMLLRGANGVAYSSLPDNAIDHFVKQAKDNGVDIFRVFDALNDLEQLKVGVDAVKKAGGVVEATVCFSGDMLQPGKKYNLDYYLEIAEKIVQMGTHILGIKDMAGTMKPAAAKLLIGSLRAKYPDLPIHVHTHDSAGTAVASMTACALAGADVVDVAINSMSGLTSQPSINALLASLEGNIDTGINVEHVRELDAYWAEMRLLYSCFEADLKGPDPEVYQHEIPGGQLTNLLFQAQQLGLGEQWAETKRAYREANYLLGDIVKVTPTSKVVGDLAQFMVSNKLTSDDVRRLANSLDFPDSVMDFFEGLIGQPYGGFPEPFRSDVLRNKRRKLTCRPGLELEPFDLEKIREDLQNRFGDVDECDVASYNMYPRVYEDFQKMRETYGDLSVLPTRSFLSPLETDEEIEVVIEQGKTLIIKLQAVGDLNKKTGEREVYFDLNGEMRKIRVADRSQKVETVTKSKADMHDPLHIGAPMAGVIVEVKVHKGSLIKKGQPVAVLSAMKMEMIISSPSDGQVKEVFVSDGENVDSSDLLVLLEDQVPVETKA

SEQ ID NO: 3 - DNA, 1374 bp, Synthetic construct
  source 1..1374: mol_type = other DNA
atgttctctgctactagaactgctgctagaacagcagctcaagctgctaatagaagaaatgtttccaccatcagagaaaccttgaaagaaatcatcccagaaagacaagccttgttgaagcaaatcaaatctgaacatggtgctaagaccttgggtaatgttactgttgaacaagttgttggtggtggtagaggtattaagtttatgttgtgggatccatccgttttggatgctgaagaaggtattagattttggggtagatctatccctgaatgccaaagagatttgccaactgctccaggtggtcaagaaattttgccagaagctatgttttggtacttgttgactggtaaagttccaaccgctgaacaaactaagcaattccaagaagaattggtttccagatctgaattgccagctcatgttgaaaaggttttggattctttgccaaagaccttgcatccaatgacccaatttgttattggtgttgctgctttgaaccacgattctcaatttgctgcaagatacaaagctggtatgaagaaagctgaatattgggaaccagcattggaagattctttggattgtgttgctaagtccttcactattgcagctagaatctacttgcactcttacaaagatggtgctgcaaatatggctccagttgataagacaaaagatttgtctgctaacttcgccactcaaatcggttttggtgattctgaaggtttcgtcgaattgatcagattatacaactccttgcataccgatcatgaaggtggtaatgtttctgctcataccactcatttggttggttctgctttgtctgatccattcttgtcttattctgctgctttaggtggtttggctggtccattgcatggtttagctaatcaagaagtcttgagattcatcttgggtatgcaaaaagaattgggtgattccccatctgatgaacaaatcgttcaatatatctggaaaactttgaactccggtcaagttattccaggttatggtcatgctgttttgagaaaaccagatcctagatttgctgccttgagagaatttggtaacaaacatccagaaaccgctaacgatccagttttcagaatggttgattccttgtttaaagttgccccaggtgttttgacagaacatggtaaaactaagaacccattcccaaatgttgatgctgcttctggttctttgttgtatcattacggtttgactcaattcccttactacactgttactttcggtacttctagagctattggtgctttgtcacaatacgtttgggatagagctttgggtttgccaattgaaagaccaaagtctttgtccatggaagccattttgaagttggtcaagtga

SEQ ID NO: 4 - DNA, 3651 bp, Synthetic construct
  source 1..3651: mol_type = other DNA
atgctttcactacgtcaatctataagatttttcaagccagccacaagaactttgtgtagctctagatatctgcttatgtcaggcggaggtggcatgtcgcaaagaaaattcgccatgtcgcaaagaaaattcgccggcttgagagataacttcaatctcttgggtgaaaagaacaaaatattggtggctaatagaggagaaattccaatcagaatttttcgtaccgctcatgaactgtctatgcagacggtagctatatattctcatgaagatcgtctttcaacgcacaaacaaaaggctgacgaagcatacgtcataggtgaagtaggccaatatacccccgtcggcgcttatttggccattgacgaaatcatttccattgcccaaaaacaccaggtagatttcatccatccaggttatgggttcttgtctgaaaattcggaatttgccgacaaagtagtgaaggccggtatcacttggattggccctccagctgaagttattgactccgtgggtgataaggtctcagctagaaacctggcagcaaaagctaatgtgcccaccgttcctggtacaccaggtcctatagaaactgtagaggaagcacttgacttcgtcaatgaatacggctacccggtgatcattaaggccgcctttggtggtggtggtagaggtatgagagtcgttagagaaggtgacgacgtggcagatgcctttcaacgtgctacctccgaagcccgtactgccttcggtaatggtacctgctttgtggaaagattcttggacaagccaaagcatattgaagttcaattgttggccgataaccacggaaacgtggttcatcttttcgaaagagactgttccgtgcagagaagacaccaaaaggttgtcgaagtggccccagcaaagactttaccccgtgaagtccgtgacgccattttgacagatgcagttaaattggccaaagagtgtggctacagaaatgcgggtactgctgaattcttggttgataaccaaaatagacactatttcattgaaattaatccaagaatccaagtggaacataccatcacagaagaaattaccggtatagatattgtggcggctcagatccaaattgcggcaggtgcctctctaccccagctgggcctattccaggacaaaattacgactcgtggctttgccattcagtgccgtattaccacggaagaccctgctaagaacttccaaccagataccggtagaatagaagtgtaccgttctgcaggtggtaatggtgttagactggatggtggtaacgcctatgcaggaacaataatctcacctcattacgactcaatgctggtcaaatgctcatgctccggttccacctacgaaatcgttcgtagaaaaatgattcgtgcattaatcgagttcagaattagaggtgtcaagaccaacattcccttcctattgactcttttgaccaatccagtatttattgagggtacatactggacgacttttattgacgacaccccacaactgttccaaatggtttcatcacaaaacagagcccaaaaacttttacattacctcgccgacgtggcagtcaatggttcatctatcaagggtcaaattggcttgccaaaattaaaatcaaatccaagtgtcccccatttgcacgatgctcagggcaatgtcatcaacgttacaaagtctgcaccaccatccggatggaggcaagtgctactagaaaaggggccagctgaatttgccagacaagttagacagttcaatggtactttattgatggacaccacctggagagacgctcatcaatctctacttgcaacaagagtcagaacccacgatttggctacaatcgctccaacaaccgcacatgcccttgcaggtcgtttcgccttagaatgttggggtggtgccacattcgatgttgcaatgagatttttgcatgaggatccatgggaacgtttgagaaaattaagatctctggtgcctaatattccattccaaatgttattgcgtggtgccaatggtgtggcttattcttcattgcctgacaatgctattgaccatttcgtcaagcaagccaaggataatggtgttgatatatttagagtctttgatgccttaaatgacttggaacaattgaaggtcggtgtagatgctgtgaagaaggcaggtggtgttgtagaagccactgtttgtttctctggggatatgcttcagccaggcaagaaatacaatttggattactacttggaaattgctgaaaaaattgtccaaatgggcactcatatcctgggtatcaaagatatggcaggtaccatgaagccagcagctgccaaactactgattggatctttgagggctaagtaccctgatctcccaatacatgttcacactcacgattctgcaggtactgctgttgcatcaatgactgcgtgtgctctggcgggcgccgatgtcgttgatgttgccatcaactcaatgtctggtttaacttcacaaccatcaatcaatgctctgttggcttcattagaaggtaatattgacactggtattaacgttgagcatgtccgtgaactagatgcatattgggcagagatgagattgttatactcttgtttcgaggctgacttgaagggcccagatccagaagtttatcaacatgaaatcccaggtggtcaattgacaaacttgttgtttcaagcccaacaattgggtcttggagaacaatgggccgaaacaaaaagagcttacagagaagccaattatttattgggtgatattgtcaaagttaccccaacttcgaaggtcgttggtgatctggcacaatttatggtctccaataaattaacttccgatgatgtgagacgcctggctaattctttggatttccctgactctgttatggatttcttcgaaggcttaatcggccaaccatatggtgggttcccagaaccatttagatcagacgttttaaggaacaagagaagaaagttgacttgtcgtccaggcctggaactagagccatttgatctcgaaaaaattagagaagacttgcagaatagatttggtgatgttgatgagtgcgacgttgcttcttataacatgtacccaagagtttatgaagacttccaaaagatgagagaaacgtatggtgatttatctgtattgccaacaagaagctttttgtctccactagagactgacgaagaaattgaagttgtaatcgaacaaggtaaaacgctaattatcaagctacaggctgtgggtgatttgaacaaaaagaccggtgaaagagaagtttactttgatttgaatggtgaaatgagaaaaattcgtgttgctgacagatcacaaaaagtggaaactgttactaaatccaaagcagacatgcatgatccattacacattggtgcaccaatggcaggtgtcattgttgaagttaaagttcataaaggatcactaataaagaagggccaacctgtagccgtattaagcgccatgaaaatggaaatgattatatcttctccatccgatggacaagttaaagaagtgtttgtctctgatggtgaaaatgtggactcttctgatttattagttctattagaagaccaagttcctgttgaaactaaggcatga

SEQ ID NO: 5 - protein, 655 aa, Aspergillus nidulans
MPAAPLVSTANGPNANDNITRFEPPSRVRSPFADALFHNKTRCFVYGMQPRAVQGMLDFDFICKRSTPSVAGIIYTFGGQFVSKMYWGTSETLLPVYQDTAKAMAKHPDVDTVVNFASSRSVYSSTMELMQYPQIKCIAIIAEGVPERRAREILVTAKEKGITIIGPATVGGIKPGAFKIGNTGGMMDNIVASKLYRKGSVGYVSKSGGMSNELNNIISQTTDGVYEGVAIGGDRYPGTTFIDHLLRYQAEPECKILVLLGEVGGVEEYRVIEAVKNGVITKPIVAWAIGTCASMFKTEVQFGHAGASANSDLETAVAKNKAMREAGIYVPDTFEDMPAVLKKVYEEQVQNGVIKPQPEPVPPKIPIDYSWAQELGLIRKPAAFISTISDDRGQELLYAGMPISDVFKEDIGIGGVMSLLWFRRRLPSYATKFLEMVLMLTADHGPAVSGAMNTIITTRAGKDLISALVSGLLTIGSRFGGALDGAAEEFTKAFDKGMSPRDFVDTMRKENKLIPGIGHRIKSRNNPDLRVELVKEYVKKHFPSTKLLDYAIAVETVTTSKKDNLILNVDGCIAVCFVDLMRNCGAFSAEESEDYMKMGVLNGLFVLGRSIGLIAHYLDQKRLRTGLYRHPWDDITYLLPALQKGGSEGRVEVNV

SEQ ID NO: 6 - DNA, 1287 bp, Saccharomyces cerevisiae
  source 1..1287: mol_type = genomic DNA
atgagtatgttatctagaagattattttccacctctcgccttgctgctttcagtaagattaaggtcaaacaacccgttgtcgagttggacggtgatgaaatgacccgtatcatttgggataagatcaagaagaaattgattctaccctacttggacgtagatttgaagtactacgacttatctgtcgaatctcgtgacgccacctccgacaagattactcaggatgctgctgaggcgatcaagaagtatggtgttggtatcaaatgtgccaccatcactcctgatgaagctcgtgtgaaggaattcaacctgcacaagatgtggaaatctcctaatggtaccatcagaaacattctcggcggtacagtgttcagagagcccattgtgattcctagaattcctagactggtcccacgttgggaaaaaccaatcattattggaagacacgcccacggtgatcaatataaagctacggacacactgatcccaggcccaggatctttggaactggtctacaagccatccgaccctacgactgctcaaccacaaactttgaaagtgtatgactacaagggcagtggtgtggccatggccatgtacaatactgacgaatccatcgaagggtttgctcattcgtctttcaagctggccattgacaaaaagctaaatcttttcttgtcaaccaagaacactattttgaagaaatatgacggtcggttcaaagacattttccaagaagtttatgaagctcaatataaatccaaattcgaacaactagggatccactatgaacaccgtttaattgatgatatggtcgctcaaatgataaaatctaaaggtggctttatcatggcgctaaagaactatgacggtgatgtccaatctgacatcgtcgctcaaggatttggctccttaggtttgatgacttctatcttagttacaccagacggtaaaactttcgaaagtgaagctgctcatggtaccgtgacaagacattatagaaagtaccaaaagggtgaagaaacttctacaaactccattgcatccattttcgcgtggtcgagaggtctattgaagagaggtgaattggacaatactcctgctttgtgtaaatttgccaatattttggaatccgccactttgaacacagttcagcaagacggtatcatgacgaaggacttggctttggcttgcggtaacaacgaaagatctgcttatgttaccacagaagaatttttggatgccgttgaaaaaagactacaaaaagaaatcaagtcgatcgagtaa

SEQ ID NO: 7 - protein, 563 aa, Saccharomyces cerevisiae
MSEITLGKYLFERLKQVNVNTVFGLPGDFNLSLLDKIYEVEGMRWAGNANELNAAYAADGYARIKGMSCIITTFGVGELSALNGIAGSYAEHVGVLHVVGVPSISAQAKQLLLHHTLGNGDFTVFHRMSANISETTAMITDIATAPAEIDRCIRTTYVTQRPVYLGLPANLVDLNVPAKLLQTPIDMSLKPNDAESEKEVIDTILALVKDAKNPVILADACCSRHDVKAETKKLIDLTQFPAFVTPMGKGSIDEQHPRYGGVYVGTLSKPEVKEAVESADLILSVGALLSDFNTGSFSYSYKTKNIVEFHSDHMKIRNATFPGVQMKFVLQKLLTTIADAAKGYKPVAVPARTPANAAVPASTPLKQEWMWNQLGNFLQEGDVVIAETGTSAFGINQTTFPNNTYGISQVLWGSIGFTTGATLGAAFAAEEIDPKKRVILFIGDGSLQLTVQEISTMIRWGLKPYLFVLNNDGYTIEKLIHGPKAQYNEIQGWDHLSLLPTFGAKDYETHRVATTGEWDKLTQDKSFNDNSKIRMIEIMLPVFDAPQNLVEQAKLTAATNAKQ

SEQ ID NO: 8 - protein, 563 aa, Saccharomyces cerevisiae
MSEITLGKYLFERLSQVNCNTVFGLPGDFNLSLLDKLYEVKGMRWAGNANELNAAYAADGYARIKGMSCIITTFGVGELSALNGIAGSYAEHVGVLHVVGVPSISSQAKQLLLHHTLGNGDFTVFHRMSANISETTAMITDIANAPAEIDRCIRTTYTTQRPVYLGLPANLVDLNVPAKLLETPIDLSLKPNDAEAEAEVVRTVVELIKDAKNPVILADACASRHDVKAETKKLMDLTQFPVYVTPMGKGAIDEQHPRYGGVYVGTLSRPEVKKAVESADLILSIGALLSDFNTGSFSYSYKTKNIVEFHSDHIKIRNATFPGVQMKFALQKLLDAIPEVVKDYKPVAVPARVPITKSTPANTPMKQEWMWNHLGNFLREGDIVIAETGTSAFGINQTTFPTDVYAIVQVLWGSIGFTVGALLGATMAAEELDPKKRVILFIGDGSLQLTVQEISTMIRWGLKPYIFVLNNNGYTIEKLIHGPHAEYNEIQGWDHLALLPTFGARNYETHRVATTGEWEKLTQDKDFQDNSKIRMIEVMLPVFDAPQNLVKQAQLTAATNAKQ

SEQ ID NO: 9 - protein, 563 aa, Saccharomyces cerevisiae
MSEITLGKYLFERLKQVNVNTIFGLPGDFNLSLLDKIYEVDGLRWAGNANELNAAYAADGYARIKGLSVLVTTFGVGELSALNGIAGSYAEHVGVLHVVGVPSISAQAKQLLLHHTLGNGDFTVFHRMSANISETTSMITDIATAPSEIDRLIRTTFITQRPSYLGLPANLVDLKVPGSLLEKPIDLSLKPNDPEAEKEVIDTVLELIQNSKNPVILSDACASRHNVKKETQKLIDLTQFPAFVTPLGKGSIDEQHPRYGGVYVGTLSKQDVKQAVESADLILSVGALLSDFNTGSFSYSYKTKNVVEFHSDYVKVKNATFLGVQMKFALQNLLKVIPDVVKGYKSVPVPTKTPANKGVPASTPLKQEWLWNELSKFLQEGDVIISETGTSAFGINQTIFPKDAYGISQVLWGSIGFTTGATLGAAFAAEEIDPNKRVILFIGDGSLQLTVQEISTMIRWGLKPYLFVLNNDGYTIEKLIHGPHAEYNEIQTWDHLALLPAFGAKKYENHKIATTGEWDALTTDSEFQKNSVIRLIELKLPVFDAPESLIKQAQLTAATNAKQ

SEQ ID NO: 10 - DNA, 1077 bp, Saccharomyces cerevisiae
  source 1..1077: mol_type = genomic DNA
atgtttgtttcaccaccaccagcaacttcgaaaaaccaagttttacaacgacgtccattagaatcgactaacagtaatcatgggtttgcaagctccctacaggccattccggaaaacacgatgagtggcagtgataatgcttcttttcaaagtttgccactatcaatgttttctgccccctctactgtgcacacgcaactaactaatgactcttcgttctccgaatttcctaaccacaagttaatcacgagagtgagcctggatgaagcattacccaaaacgttttatgacatgtattcgccagatattctattagcagacccatccaacattctctgtaacgggcgtcccaagtttaccaagagagagttattggattgggatttaaacgatataagatcgttattgatagtcgagaagttaaggcccgaatggggtaatcaactaccggaagtaataacggtgggtgataatatgccccagtttaggttacaattattaccactatattctagcgatgagaccataatcgcaacgttagtccattcggatctgtacatggaggctaacttagattatgaattcaaactaaccagcgccaaatatacagtagcgaccgctagaaaaagacatgagcatataactggtagaaatgaagccgtcatgaatttgtcgaaaccggaatggagaaatatcatcgaaaattacctcttaaatatagcagtagaggcacaatgcaggtttgatttcaaacaaagatgctccgaatataagaaatggaagttacaacagtccaacttaaaaagaccggacatgcccccaccaagcataataccgcggaaaaacagcacagaaacaaaatcgcttctgaaaaaggctttattgaagaacattcagttgaaaaaccccaataataaccttgatgaattgatgatgagatcaagcgccgcaacaaatcaacagggaaaaaacaaagtcagcttatctaaagaagaaaaggctacgatatggtcgcaatgtcaggcacaagtttaccaaagattaggattggattggcagccggattcagtatcctga

SEQ ID NO: 11 - protein, 500 aa, Saccharomyces cerevisiae
MSRLERLTSLNVVAGSDLRRTSIIGTIGPKTNNPETLVALRKAGLNIVRMNFSHGSYEYHKSVIDNARKSEELYPGRPLAIALDTKGPEIRTGTTTNDVDYPIPPNHEMIFTTDDKYAKACDDKIMYVDYKNITKVISAGRIIYVDDGVLSFQVLEVVDDKTLKVKALNAGKICSHKGVNLPGTDVDLPALSEKDKEDLRFGVKNGVHMVFASFIRTANDVLTIREVLGEQGKDVKIIVKIENQQGVNNFDEILKVTDGVMVARGDLGIEIPAPEVLAVQKKLIAKSNLAGKPVICATQMLESMTYNPRPTRAEVSDVGNAILDGADCVMLSGETAKGNYPINAVTTMAETAVIAEQAIAYLPNYDDMRNCTPKPTSTTETVAASAVAAVFEQKAKAIIVLSTSGTTPRLVSKYRPNCPIILVTRCPRAARFSHLYRGVFPFVFEKEPVSDWTDDVEARINFGIEKAKEFGILKKGDTYVSIQGFKAGAGHSNTLQVSTV

SEQ ID NO: 12 - protein, 506 aa, Saccharomyces cerevisiae
MPESRLQRLANLKIGTPQQLRRTSIIGTIGPKTNSCEAITALRKAGLNIIRLNFSHGSYEFHQSVIENAVKSEQQFPGRPLAIALDTKGPEIRTGRTLNDQDLYIPVDHQMIFTTDASFANTSNDKIMYIDYANLTKVIVPGRFIYVDDGILSFKVLQIIDESNLRVQAVNSGYIASHKGVNLPNTDVDLPPLSAKDMKDLQFGVRNGIHIVFASFIRTSEDVLSIRKALGSEGQDIKIISKIENQQGLDNFDEILEVTDGVMIARGDLGIEILAPEVLAIQKKLIAKCNLAGKPVICATQMLDSMTHNPRPTRAEVSDVGNAVLDGADCVMLSGETAKGDYPVNAVNIMAATALIAESTIAHLALYDDLRDATPKPTSTTETVAAAATAAILEQDGKAIVVLSTTGNTARLLSKYRPSCPIILVTRHARTARIAHLYRGVFPFLYEPKRLDDWGEDVHRRLKFGVEMARSFGMVDNGDTVVSIQGFKGGVGHSNTLRISTVGQEF

SEQ ID NO: 13 - protein, 585 aa, Saccharomyces cerevisiae
MSHPHSHSIYLSELPVRKPQALGNPLLRKIQRACRMSLAEPDLALNLDIADYINEKQGAAPRDAAIALAKLINNRESHVAIFALSLLDVLVKNCGYPFHLQISRKEFLNELVKRFPGHPPLRYSKIQRLILTAIEEWYQTICKHSSYKNDMGYIRDMHRLLKYKGYAFPKISESDLAVLKPSNQLKTASEIQKEQEIAQAAKLEELIRRGKPEDLREANKLMKIMAGFKEDNAVQAKQAISSELNKLKRKADLLNEMLESPDSQNWDNETTQELHSALKVAQPKFQKIIEEEQEDDALVQDLLKFNDTVNQLLEKFNLLKNGDSNAASQIHPSHVSAPLQQSSGALTNEINLIDFNDLDEAPSQGNNNTNGTGTPAAAETSVNDLLGDLTDLSISNPSTANQASFGLGGDIVLGSSQPAPPVTTTNNSNNTLDLLGLSTPQSPTNSQAVNSSGFDLLMGFNPTTGTTTAPARTLVNQSPNLKIEFEISRESNSVIRIKSFFTNLSSSPISNLVFLLAVPKSMSLKLQPQSSNFMIGNAKDGISQEGTIENAPANPSKALKVKWKVNYSVNSTQAEETAVFTLPNV

SEQ ID NO: 14 - protein, 369 aa, Saccharomyces cerevisiae
MLRNTFFRNTSRRFLATVKQPSIGRYTGKPNPSTGKYTVSFIEGDGIGPEISKSVKKIFSAANVPIEWESCDVSPIFVNGLTTIPDPAVQSITKNLVALKGPLATPIGKGHRSLNLTLRKTFGLFANVRPAKSIEGFKTTYENVDLVLIRENTEGEYSGIEHIVCPGVVQSIKLITRDASERVIRYAFEYARAIGRPRVIVVHKSTIQRLADGLFVNVAKELSKEYPDLTLETELIDNSVLKVVTNPSAYTDAVSVCPNLYGDILSDLNSGLSAGSLGLTPSANIGHKISIFEAVHGSAPDIAGQDKANPTALLLSSVMMLNHMGLTNHADQIQNAVLSTIASGPENRTGDLAGTATTSSFTEAVIKRL

SEQ ID NO: 15 - DNA, 904 bp, Saccharomyces cerevisiae
  source 1..904: mol_type = genomic DNA
aacctagattgctatctccactccctatgcttgctttggtggactattgtttccgttattcgcaatttctataggactactatagattatataaactgaattttagctttatgctgtggtgttatttcccaaaacacgctcttctttcttacgctttccgttagcttatttgctcacggtatcaagattacttttttttttttcttcttttctttttcttgcactaccctcttcttcttgttcttccttgcgtttttacccctcttccctttattttgcagcaaaaagaaagatggaaaggcaaaagtgaaggcatgatgatgacaaacttgaaaagaaagagaacagcgaaaagagatgaataatccatggcaatgactgcacgctcatgggtatcaattggctaggtctaatattgttattgtttggagtatggcgtagagaagtggttccttaaccttaattaatgcccgtgccatgatgattgcatcactgagacgtaatgtggaaacaccacttctcggtccgcggacatcaaccgaaggaaaaatgacaagctatttccgtgtatatccgcgccaatcccttcggggccgaagttcggaggctttatctccgcttagccaaggccgtcaaaggcgataaggtgcgttgcaccccaattagccatggagaaggaaaaaaaaagcatgtatgatcatgatgatgcaaaaattgagaggaaaaatgactcaggctatttatataagtaaatgaaagggtttcgttgtagccttttctgctctcttccttcgctcttccgcatatatatttgtgttcagagattattcttaaatccataagaacatcccttcatataacaattgaataaggaaaacacaacacataacacatatttaacctg

SEQ ID NO: 16 - DNA, 999 bp, Saccharomyces cerevisiae
  source 1..999: mol_type = genomic DNA
tttgtcgcccttgagtaaaagaaagcgacaaggtgtgcaattgttatgagtaggattcagttataattatcacttagcatttgttctaaacatgaacacacaacgaatgcctgtaaacacgcttgccgcgctagacatgcaaacgatcctcaaaaaaaaaaaaacgctcgtatgaagacctaatttacgtatttttttttttttgtttttcgtttacgcgttaattgatcgattatgagcttttggggaaaagtaattgccgcagcggctgtcgtaccggaagagtgttgtttttcattaccaaaaagggataatagtggttcctttatgtgacattggtaagctgattgggcataattacttgcctgatgacgaataacgcgagaactgcccatatagaatctgacgacgtaaggagaagaaaaagcgggaaacggatttctcgttctaggcctcggcggctcttaaaattgtaaaatgaaaatgagaataaaattaaactatacacagctgatcagcgttctaatgaaaaaaaaatgcattggaaggaaaagccgaaatacttcattttctgggcattgacgttctgttgcctcttgtgcatacacacttgtttttattgaaatcgagcttattgtaagagaacttgcgcttatcttcaaatgccgctttcctcgaaaaagcatttaaacatcattcaagattattagtctatcaagcttgtccagactgcttgttattatgtaaaacaagtatgttggtattgtatttgattgccgattctcatatcgccatacacgaaccaattatgcatcataaaaaaagcgtagttcttttacccgaccaaccaacttcttcctttgtcctcaatatcaaagaaaaaaaaaaaaacccactgctcagatgttataaggaaggggtgttaacttatatacaggttcatctaccagtcaccagtccatacaaacttgaaccgtctgcgtaccagtcctaatcaaa

SEQ ID NO: 17 - DNA, 457 bp, Saccharomyces cerevisiae
  source 1..457: mol_type = genomic DNA
gcttctcattctattttaattatactagtacgatttctcactctgtaatttaatatcagtgtaatatgcacctagttatgggtagtttttgctaacgttacgagccgcgaaactgtcctcaatcttcaccactacctctaatgactgaagaatgctatgcgatataacgctgtcgcactttgaatatatacttatatttacatagttttcaagtgcgtattactattgcaaagtagtattttgtcacgtgattttgatccaattaaaactaaatatggttcaacccgttgtttccgcatcaaaaaaccataccatttatcaaggggacgggatatatcacataacagtttgaatgcataatttgttatagatatcttctggaataatcttcacagcaaaagcgcaagtcgaataatatatcgataaatacaatccataagacttaaaactaacctca

SEQ ID NO: 18 - DNA, 438 bp, Saccharomyces cerevisiae
  source 1..438: mol_type = genomic DNA
gctgggcgatcttccttgtgcgtctgttgtctcacaattgcttgaaggaagatttcataagatcatatgagtccctctttatatgggcaagtggaattatgtgagtcaaaatccgcgcgtgacccgtaaagcgttatcagaaggtgcaaacggtgctatttagctcataaaaggaatgattcaagctcttttggattgtaagacacctttatttagtccaagatcattgcagacccttgttatggtctttagcagagtcctcctctatatctcttcatttactgcaacctgattggcccgctaccacgatgcccgctttgttcctgtggtattaaaagaatcgatgaaagagactcttatcttcagggaaaattaggaccgagaattagagcaagcaagatatttgcaaactactaactacaagcgacttacacagac

SEQ ID NO: 19 - DNA, 546 bp, Saccharomyces cerevisiae
  source 1..546: mol_type = genomic DNA
tcattctaccatctttcatgtccctacgccattctcgttcgtataccatataaaccaattatagattttacatagaataatagttaaatttgattatagaatatcttcggattcaataaaaaattattattatcagactctttctgtccactccttgaagtatgcaaagtgcttcgagaacgccatgacattcgacaattcaaaattgagagaggactgctaaggaaactctttgagttgattatataatcgctatttaatcttttaaagggggaatactgctaaattttcattattgtacaactggagacatgataggcctgaaatgtgccaccaaattgcatgaccttgctaatgaggtcgctgggttgcgtatgcactttttaccctgctccgcgtcgaaacagaacataatacagagtaaagaggaaagagcgaaaatgaaaaagtttctacactcaaaagtcaaacctttttaaaaagtgatgttcagattccttgtttaagacaaataccattgaggaaggcgattgaccctaacgaagt

SEQ ID NO: 20 - DNA, 1000 bp, Saccharomyces cerevisiae
  source 1..1000: mol_type = genomic DNA
ggccacaatgaaacttcaattcatatcgaccgactatttttctccgaaccaaaaaaatagcagggcgagattggagctgcggaaaaaagaggaaaaaattttttcgtagttttcttgtgcaaattagggtgtaaggtttctagggcttattggttcaagcagaagagacaacaattgtaggtcctaaattcaaggcggatgtaaggagtattggtttcgaaagtttttccgaagcggcatggcagggactacttgcgcatgcgctcggattatcttcatttttgcttgcaaaaacgtagaatcatggtaaattacatgaagaattctcttttttttttttttttttttttttttacctctaaagagtgttgaccaactgaaaaaacccttcttcaagagagttaaactaagactaaccatcataacttccaaggaattaatcgatatcttgcactcctgatttttcttcaaagagacagcgcaaaggattatgacactgttgcattgagtcaaaagtttttccgaagtgacccagtgctcttttttttttccgtgaaggactgacaaatatgcgcacaagatccaatacgtaatggaaattcggaaaaactaggaagaaatgctgcagggcattgccgtgccgatcttttgtctttcagatatatgagaaaaagaatattcatcaagtgctgatagaagaataccactcatatgacgtgggcagaagacagcaaacgtaaacatgagctgctgcgacatttgatggcttttatccgacaagccaggaaactccaccattatctaatgtagcaaaatatttcttaacacccgaagttgcgtgtccccctcacgtttttaatcatttgaattagtatattgaaattatatataaaggcaacaatgtccccataatcaattccatctggggtctcatgttctttccccaccttaaaatctataaagatatcataatcgtcaactagttgatatacgtaaaatc

SEQ ID NO: 21 - DNA, 455 bp, Saccharomyces cerevisiae
  source 1..455: mol_type = genomic DNA
cgttcccaaaccattacattatgttctttcacttattacatacgatatatagcatctacatctacatatctaaatgatgttttttcccatctactttgtccgatacgcattaaaagaagaagatttactctcccgtgatgccgggccaatcagacgcgcgcattcgcggcagaataaggggaggctgctgcgggtaccaaatatccagatattcacaattatgccaatcaactgtctagttgctgacacccatcgttttccactgcaacgaggttttggggctagaaaaggcgttaacaatcgttagagaaggaggggttcataacttgacttgttgtatcttgtttaaaactttctttgaatttgttcgtggttgaatttgtttttgctatcccaaaggagtgcattttacacgcattactacagcacacttttatacagttccacaatagaat

SEQ ID NO: 22 - DNA, 424 bp, Saccharomyces cerevisiae
  source 1..424: mol_type = genomic DNA
atagataaaaaaaaaacgcaccaagtaagtaagtaaataaagaataaataaactatatgagtaaaacaccaagcgaggatgtttcattgtgcatccgtgttcttgatgatcacataactgtaaaagaataatacggcacgttaaatgttattttagaatatataaacaccttatgtgccataagcattgagccaatcgctgctgttttttttattccggggcaccttcggaagaacacaggcgcaatttagttatataaggagaagccctcgagcgatcaggggaccgactgcggatcgctttaaggcaaagatagaaggataaatatctgctttggaagatagtcgtatctaatttcccattctgttgttttcttgatctttcctacgctttcgactttcttcctacgcgctttataatagct

SEQ ID NO: 23 - DNA, 812 bp, Saccharomyces cerevisiae
  source 1..812: mol_type = genomic DNA
ctttttttgctctatatttattggtaacagtgcttattgttactcaatgtatggtacgctgcatacctatcaagatgcgactacatatatacaattatctcatctcatcgcgtattgaaaaattttccatcttctaatttttcagaacccataaattcgggtaacgatcagtggaaaaaacttaaatccgcagtaataggcttgcaaaggaccaaagacagcatatgcagcagttaagaaatctaagaatgcattgtatattagagtagtatatggaaaagctcggactgggagccgtaaaatctctttcccgtaagatctggctgcaaacctggtctacataaatgtagtacattataagggactaccaagaaatacgaagcgctcagatatctcttaaagttcatttttttgccggaataatcttggtttagtaacttataaaactacttacgtccaataaatcgttatattttatgccgtcgtcataaagtggacactttcgagagacggtgctggccaataagatgctgatatttgaaatatcatttacccgggcccgggtgggaaaaacaaggaattaaagataatgaagtgatcttttggcgagatagtaaaactttcaattatctaggaacaatttcaaacgccaattgttaactttgtgtacagtatttctagtttttatctcattgaaaacaaaattcagtcaacttggaaatttaagcaggctactgtggcattgagatatttttccgcagagtaagaaccgattagcaagttaccgcattatttttaaaccgtggacaatc

SEQ ID NO: 24 - DNA, 1000 bp, Saccharomyces cerevisiae
  source 1..1000: mol_type = genomic DNA
tttgatggaagacgagggtaatgaagaatacaccaaagatttggaagaagcagctaacaaagctcaaccacagtagaatggaatattgaaactggagtgacttctgatgaagtttccaattatttatatgtatacattatttatttgtctatcattaaaatttctgtcaagtaatggctttttatgtttatttcatggattacgaaatttgctggctttttagagtgaccgatgagttgcatgtacatatgtgcgaaataaaacaatacggtagtaaaacatgaatacttcgagaagtaaattcaagattgcaggcgtagcacaagaatgtccttccgcctgctagtgtgttgagcaaagcacgccatcactaatggaacccttacaaaatctcgcatggagcagcaaaccttaaatccattttctatcattcgttcctgaggacccggatatcgttttagagacaaaaatagatactacaagaggaatagggatatctgcacatggggcctcgagcatggctcattttcgattttccggcacgatgctgacttagtactatcatgccaggcaggacaggccttatggggttcttgcagcttaccaattctgacactggactgcccctggattagcgaccccgcgaccccgcgaccagactccctgaaaatggtaattggtgatgatatatgtgactgagttcagcctgggcatacaatgtttaacccttttggacatggaattaggccagcaaagggagcagctttccccgaaagcgtagtttttctccaggaacgcaattcacccctttctttcttatgcaagctcctcgtacatatataaaaatatatgagtatatatagtgcttctacagcataatcaatttaaggttccttgcctttccttaaatatgttagctatctctaaagcatcactgagaagtggtagtttttttgataactgtgattgaagttttgactacctcagagaaaaattttga

SEQ ID NO: 25 - DNA, 1000 bp, Saccharomyces cerevisiae
  source 1..1000: mol_type = genomic DNA
aagatctgttacgtagtatttgagagaaggagtaaactaaccttgaattgaggtcttgattacaagttgcttgcttagtgacacatttgacaagaaaaaacagtaatacgccagaacatttgcggcccctttttatatcaaaagtgctaaacatgctatttaagtatttcttttgctgttgatacgcaattttcattgcgctgctgcagatgtggtaccctttaccgcacaaaaatacttctatctggatattattccgtgtacttggcgaagaagccatcccgttttgaacgccattgccgctaacctccgcgctttcgcttgcagcaatggctacatccgcacggttccgctattgccacgacattgtggtggtaatagccaaggcaatggtgtgtacaacttacgatccctctgcttgcggaagcagtcaaaaggcaagtgagatctactctataaactgacactttatccagggaattatcgagaaaattctgtcttgtcaattagagaagtaaggggccacagggaaataatactttcgaagggagtttctttccttaaggaggtgcaaaatgcaaaagatatacagtttataggttgatacacatagagcaagtggtctcaagtggtcttaatgagtcacgggaggtcaagatgctctttttttcttcatcgtttcagttttgtatcttgagcggagtgaacttgccctttaagcgggccatacccttacatggcgttagcacttattttcaaggtgaatatatacaatgtcagttacccgtttttttgtcttccgctgtataagcttgctatcattaaaagccgcatgtggcatcctgaattctgccaccccttaaatacaaaaaatagtgtaaatggtacttgatctcttctggaggaataaacaataattcttgatggaatttgagagaaagtaagtgttcggctaagcacaataaagatataaacctgtgactacctatataagttcgta

SEQ ID NO: 26 - DNA, 278 bp, Saccharomyces cerevisiae
  source 1..278: mol_type = genomic DNA
ttccaatattagatgcctgtgaaactactgtcaaacttatttgtgtaacaatactcatcgtctaatttgtccattattgttatatactctgtcataaaaaaagaaagaaaaatcttatactttcgtcgactgtgatttgaaaatctctccagatcaaacgggaataaaagggtttatatccgaaggtggccagaacaatcatgaagcaaataggataaaaatagccaataagatgtttgttgtctttcatcccaaagttccaacagcatatttcaaag

SEQ ID NO: 27 - protein, 186 aa, Saccharomyces cerevisiae [sequence omitted]

SEQ ID NO: 28 - protein, 125 aa, Saccharomyces cerevisiae
MFRSVCRISSRVAPSAYRTIMGRSVMSNTILAQRFYSANLSKDQVSQRVIDVIKAFDKNSPNIANKQISSDTQFHKDLGLDSLDTVELLVAIEEEFDIEIPDKVADELRSVGETVDYIASNPDAN

SEQ ID NO: 29 - protein, 391 aa, Saccharomyces cerevisiae
MGYFVPDSHIENLKSYKYQSEDRSLVSKYFLKPFWQRFCHIFPTWMAPNIITLSGFAFIVINVLTVFYYDPNLNTDTPRWTYFSYALGVFLYQTFDGCDGVHARRINQSGPLGELFDHSIDAINSTLSIFIFASETGMGFSYNLMLSQFAMLTNFYLSTWEEYHTHTLYLSEFSGPVEGILIVCVSLILTGIYGKQVIWHTYLFTITVGDKVIDVDTLDIVFSLAVFGLVMNALSAKRNVDKYYRNSTSSANNITQIEQDSAIKGLLPFFAYYASIALLVWMQPSFITLSFILSVGFTGAFTVGRIIVCHLTKQSFPMFNAPMLIPLCQIVLYKICLSLWGIESNKIVFALSWLGFGLSLGVHIMFMNDIIHEFTEYLDVYALSIKRSKLT

SEQ ID NO: 30 - protein, 700 aa, Saccharomyces cerevisiae
MVAQYTVPVGKAANEHETAPRRNYQCREKPLVRPPNTKCSTVYEFVLECFQKNKNSNAMGWRDVKEIHEESKSVMKKVDGKETSVEKKWMYYELSHYHYNSFDQLTDIMHEIGRGLVKIGLKPNDDDKLHLYAATSHKWMKMFLGAQSQGIPVVTAYDTLGEKGLIHSLVQTGSKAIFTDNSLLPSLIKPVQAAQDVKYIIHFDSISSEDRRQSGKIYQSAHDAINRIKEVRPDIKTFSFDDILKLGKESCNEIDVHPPGKDDLCCIMYTSGSTGEPKGVVLKHSNVVAGVGGASLNVLKFVGNTDRVICFLPLAHIFELVFELLSFYWGACIGYATVKTLTSSSVRNCQGDLQEFKPTIMVGVAAVWETVRKGILNQIDNLPFLTKKIFWTAYNTKLNMQRLHIPGGGALGNLVFKKIRTATGGQLRYLLNGGSPISRDAQEFITNLICPMLIGYGLTETCASTTILDPANFELGVAGDLTGCVTVKLVDVEELGYFAKNNQGEVWITGANVTPEYYKNEEETSQALTSDGWFKTGDIGEWEANGHLKIIDRKKNLVKTMNGEYIALEKLESVYRSNEYVANICVYADQSKTKPVGIIVPNHAPLTKLAKKLGIMEQKDSSINIENYLEDAKLIKAVYSDLLKTGKDQGLVGIELLAGIVFFDGEWTPQNGFVTSAQKLKRKDILNAVKDKVDAVYSSS

SEQ ID NO: 31 - protein, 185 aa, Saccharomyces cerevisiae
MNISGTLNTLRLLYNPSLCKPSLVVPTFNDLPIPIHDSIKAVVLDKDNCIAFPHDDKIWPDYLQHWETLRSKYSNKALLIVSNTAGSNSDKDYSQAKLLEDKTGIPVLRHSTKKPGCHNEILDYFYRNKTITNPKEVAVVGDRLFTDILMANLMGSYGVWIRDGVKVSANPLSKFEKKLYNFLGF

SEQ ID NO: 32 - protein, 420 aa, Saccharomyces cerevisiae
MSKIKVVHPIVEMDGDEQTRVIWKLIKEKLILPYLDVDLKYYDLSIQERDRTNDQVTKDSSYATLKYGVAVKCATITPDEARMKEFNLKEMWKSPNGTIRNILGGTVFREPIIIPKIPRLVPHWEKPIIIGRHAFGDQYRATDIKIKKAGKLRLQFSSDDGKENIDLKVYEFPKSGGIAMAMFNTNDSIKGFAKASFELALKRKLPLFFTTKNTILKNYDNQFKQIFDNLFDKEYKEKFQALKITYEHRLIDDMVAQMLKSKGGFIIAMKNYDGDVQSDIVAQGFGSLGLMTSILITPDGKTFESEAAHGTVTRHFRKHQRGEETSTNSIASIFAWTRAIIQRGKLDNTDDVIKFGNLLEKATLDTVQVGGKMTKDLALMLGKTNRSSYVTTEEFIDEVAKRLQNMMLSSNEDKKGMCKL

SEQ ID NO: 33 - protein, 384 aa, Saccharomyces cerevisiae
MNKTNWKVSVTTFNCGKEFPVENSKAIVKQLLFPYDDGISQLELQDLYVLGFQEVVPIWQGSFPAVNRDLIDRITTTAVNCLNEKVSATQGDEQYSCLGVNSLGAITIIVLYNNNALKVKDDILKRNGKCGWFGTHLKGGTLISFQMTRNGEENWERFSYICAHLNANEGVNNRNQRIDDYKRIMSEVCDSEVAKSDHFFFLGDLNFRVTSTYDPTTNYSSTTTLRRLLENHEELNLLRKGEDEPLCKGFQELKITFPPTYKFKLFEKETYNTKRIPSWCDRILYKSYAVPTFAQEGTYHSVPRSNALLFSDHQPVNLTVRLPRSTGTPVPLSLHIEKYPLSWSSGLIGQIGDAVIGYCGWLVTKNVHYWILGSLLLYLLLKIL

SEQ ID NO: 34 - protein, 274 aa, Saccharomyces cerevisiae
MISVMADEKHKEYFKLYYFQYMIIGLCTILFLYSEISLVPRGQNIEFSLDDPSISKRYVPNELVGPLECLILSVGLSNMVVFWTCMFDKDLLKKNRVKRLRERPDGISNDFHFMHTSILCLMLIISINAALTGALKLIIGNLRPDFVDRCIPDLQKMSDSDSLVFGLDICKQTNKWILYEGLKSTPSGHSSFIVSTMGFTYLWQRVFTTRNTRSCIWCPLLALVVMVSRVIDHRHHWYDVVSGAVLAFLVIYCCWKWTFTNLAKRDILPSPVSV

SEQ ID NO: 35 - protein, 302 aa, Saccharomyces cerevisiae
MFSRLSRSHSKALPIALGTVAIAAATAFYFANRNQHSFVFNESNKVFKGDDKWIDLPISKIEEESHDTRRFTFKLPTEDSEMGLVLASALFAKFVTPKGSNVVRPYTPVSDLSQKGHFQLVVKHYEGGKMTSHLFGLKPNDTVSFKGPIMKWKWQPNQFKSITLLGAGTGINPLYQLAHHIVENPNDKTKVNLLYGNKTPQDILLRKELDALKEKYPDKFNVTYFVDDKQDDQDFDGEISFISKDFIQEHVPGPKESTHLFVCGPPPFMNAYSGEKKSPKDQGELIGILNNLGYSKDQVFKF

SEQ ID NO: 36 - protein, 222 aa, Saccharomyces cerevisiae
MTELDYQGTAEAASTSYSRNQTDLKPFPSAGSASSSIKTTEPVKDHRRRRSSSIISHVEPETFEDENDQQLLPNMNATWVDQRGAWIIHVVIIILLKLFYNLFPGVTTEWSWTLTNMTYVIGSYVMFHLIKGTPFDFNGGAYDNLTMWEQIDDETLYTPSRKFLISVPIALFLVSTHYAHYDLKLFSWNCFLTTFGAVVPKLPVTHRLRISIPGITGRAQIS

SEQ ID NO: 37 - protein, 111 aa, Saccharomyces cerevisiae
MSTVTKYFYKGENTDLIVFAASEELVDEYLKNPSIGKLSEVVELFEVFTPQDGRGAEGELGAASKAQVENEFGKGKKIEEVIDLILRNGKPNSTTSSLKTKGGNAGTKAYN

SEQ ID NO: 38 - protein, 259 aa, Saccharomyces cerevisiae
MEPESIGDVGNHAQDDSASIVSGPRRRSTSKTSSAKNIRNSSNISPASMIFRNLLILEDDLRRQAHEQKILKWQFTLFLASMAGVGAFTFYELYFTSDYVKGLHRVILQFTLSFISITVVLFHISGQYRRTIVIPRRFFTSTNKGIRQFNVKLVKVQSTWDEKYTDSVRFVSRTIAYCNIYCLKKFLWLKDDNAIVKFWKSVTIQSQPRIGAVDVKLVLNPRAFSAEIREGWEIYRDEFWAREGARRRKQAHELRPKSE

SEQ ID NO: 39 - protein, 548 aa, Saccharomyces cerevisiae
MYFPFLGRLSITDYIIVVLVYIESIISSVLKLIPQPMINLFEWLINFSTSSDDNTIEEKLRSAPTIHEMCAIFDISVEDHLVRTEDNYILTLHRIPPISKNRFNNKVVYLHHGLLMCSDVWCCNIERHKNLPFVLHDLGYDVWMGNNRGNKYSTAHLNKPPKSNKFWDFSIDEFAFFDIPNSIEFILDITKVDKVICIGFSQGSAQMFAAFSLSEKLNRKVSHFIAIAPAMTPKGLHNRIVDTLAKSSPGFMYLFFGRKIVLPSAVIWQRTLHPTLFNLCIDIANKILFNWKSFNILPRQKIASYAKLYSTTSVKSIVHWFQILRSQKFQMFEESDNMLNSLTRPYQIANFPTRTNIKIPILLIYGGIDSLVDIDVMKKNLPFNSVFDVKVDNYEHLDLIWGKDADTLVIAKVLRFIEFFNPGNVSVKTNQLLPSASLVEELPSTTWKTTHPTHGLSYRTHSADRSPLSVQADEADEVHNADNSRFLRRVFSTSAIDEDNENEHQDDTEDQIHKEQQRRLSAYLESSKDLRQLDANSSTTALDALNKE

SEQ ID NO: 40 - protein, 274 aa, Saccharomyces cerevisiae
MIRQLNYWSRKAYLIYPFQVFVGALLSIVVSSETLNHQKETCALLKSSNIFNVIFAYKANQLWPFLFFSLAFLQIYFHYLARMDILPLPISSTETSSSYLTYTNHWPLLKNRIISIMITQYACKFVLKYLLLFLNFQFIDHVFIWTGGECSSGSKTTSAEKCRLENGKWDGGFDISGHFCFLVSISMILWMELHLFSRFVQAEDMFWVVNKWVRACLAIVCAVLVIWICILWVTAIYYHTILEKVLGCLMGFICPVFIYHILPKIGILHNYLYL